(19) United States
(12) Patent Application Publication
(10) Pub. No.: US 2020/0095590 A1
(43) Pub. Date: Mar. 26, 2020

(54) TRANSCRIPTION SYSTEM

(71) Applicant: AUTOLUS LIMITED, London (GB)

(72) Inventors: Martin Pulé, London (GB); Simon Thomas, London (GB); Shaun Cordoba, London (GB)

(21) Appl. No.: 16/470,965

(22) PCT Filed: Dec. 20, 2017
(86) PCT No.: PCT/GB2017/053835

§ 371 Date: Jun. 18, 2019

(30) Foreign Application Priority Data
Dec. 21, 2016 (GB) 1621891.9

(51) Int. Cl. C12N 15/63; C12N 15/62; C12N 15/113; C12N 15/85; A61K 35/17; C07K 14/725; C07K 14/705; C07K 16/28

(52) U.S. Cl. CPC C12N 15/635; C12N 15/62; C12N 15/113; C12N 15/85; A61K 35/17; A61K 38/00; C07K 14/70578; C07K 16/2803; C12N 2830/001; C07K 2319/30; C07K 2319/33; C07K 14/7051

(57) ABSTRACT

The present invention provides a transcription system which comprises: (a) a docking component which comprises a first binding domain; and (b) a transcription control component which comprises a transcription factor and a second binding domain which binds the first binding domain of the docking component wherein binding of the first and second binding domains is disrupted by the presence of an agent, such that in the absence of the agent the docking component and the transcription control component heterodimerize.

### FIELD OF THE INVENTION

The present invention relates to a transcription system which is controllable by means of an external agent, such as a small molecule.

### BACKGROUND TO THE INVENTION

There are a number of mechanisms by which the expression of genes in cells is controlled in vivo. It is sometimes possible to use the principles behind such endogenous mechanisms in order to artificially control gene expression in cells.

RNA Interference-Based Systems

RNA interference (RNAi) is an endogenous cellular process in which an RNA polynucleotide specifically suppresses the expression of a gene.

Small interfering RNA molecules (siRNAs) may be generated in vivo through RNase III endonuclease digestion. The digestion results in molecules that are about 21 to 23 nucleotides in length. These relatively short RNA species then mediate degradation of corresponding RNA messages and transcripts. An RNAi nuclease complex, called the RNA-induced silencing complex (RISC), helps the small dsRNAs recognize complementary mRNAs through base-pairing interactions. Following the siRNA interaction with its substrate, the mRNA is targeted for degradation by enzymes that are present in the RISC. These pathways are thought to be useful to the organisms in inhibiting viral infections, transposon jumping, and similar phenomena, and to regulate the expression of endogenous genes.

The ubiquitous presence of RNAi has prompted the development of methods and compositions for turning this natural gene regulation system into a tool for the manipulation of gene expression. An RNA polynucleotide sequence designed to correspond sufficiently to the sequence of a gene whose expression is to be suppressed (the target gene) is introduced into a cell. The presence of the appropriately designed RNA activates the RNAi pathways and result in the suppression or modulation of the target gene.

US2013096370 describes an externally controllable systemsfor manipulating the regulation of either endogenous or exogenous genes through controlled RNA interference. The system involves the use of an externally applied agent, such as a drug or other compound, to regulate expression of nucleotide sequences encoding siRNAs.

However, a disadvantage of siRNA is that it can only downregulate, not upregulate the expression of a gene. Also, it is possible that the siRNA will cross-react with other sequences resulting in the down regulation of other genes with unpredicatable effects. Finally, in order to control the expression of a plurality of genes, it is necessary for the cell to express an siRNA for each gene of interest.

Hormone Receptor-Based Systems

In order to identify the target genes for a given transcription factor, one approach which has been previously used involves fuse thing transcription factor to the ligand binding domain of, for example the glucocorticoid or estrogen receptor, to produce a system in which transcriptional activation (or repression) by the transcription factor is hormone-dependent (Superti-Furga et al PNAS 88:5114-5118). Such systems are of limited use to control transcription in vivo, however, as the hormone is likely to be ubiquitous in mammalian tissues.

Tet-Based Systems

Various tetracycline-based systems have been developed to control transcriptional transactivation through administration of an external agent. These Tet-based systems have been successfully used to control the expression of numerous transgenes in cultured cells and in whole organisms, especially in mice. The original tetracycline-controlled transcriptional activator (tTA) consists of a chimeric construct of the Escherichia coli Tn10 tetR gene and the VP16 transactivation domain (see FIG. 4a). In the absence of the inducer, doxycycline, tTA dimers specifically bind to seven tandemly repeated 19-bp tetO sequences, thereby activating transcription from a minimal promoter and driving expression of the target transgene that encodes the gene of interest. When bound to doxycycline, tTA undergoes a conformational change and cannot bind tetO sequences. In the reverse tTA (rtTA) system (shown in FIG. 4b), the tetR gene has been mutated so that it binds tetO sequences and activates transcription only in the presence of doxycycline, giving a convenient control over the target transgene.

It is therefore possible to turn transcription on and off using Tet-based systems. However, in order to control transcription of a gene using a Tet-based system in a cell, it is necessary to engineer the or each target gene in the cell to include the seven tandemly repeated 19-bp tetO sequences, upstream of a minimal promoter.

There is therefore a need for an alternative mechanism to control gene expression in an external manner which is not associated with the disadvantages of the systems mentioned above.

### DESCRIPTION OF THE FIGURES

FIG. 1—Schematic diagram illustrating the first embodiment of the invention in which transcription factor-mediated control is switched ON with an agent such as a small molecule. The docking component comprises a membrane localisation domain so that in the absence of the agent the transcription component is held on the intracellular side of the plasma membrane (A); whereas in the presence of the agent (B) the transcription component dissociates from the docking component and, as it comprises a nuclear localisation signal, translocates to the nucleus where the transcription factor binds DNA and regulates the transcription of a gene. MYR=Myristylation signal; TM=Trans-membrane; SMBP=Small molecule binding protein; M=Mimic/blocker; TF=Transcription factor; NLS=Nuclear localization signal; SM=Small molecule; NES=Nuclear export signal.

FIG. 2—Schematic diagram illustrating the second embodiment of the invention in which transcription factor-mediated control is switched OFF with an agent such as a small molecule. The docking component comprises a nuclear localisation signal so that in the absence of the agent (a) the transcription component is held in the nwhereas in the presence of the agent the transcription component dissociates from the docking component and, as it comprises a nuclear export signal is translocates to the cytoplasm, causing transcription-factor mediated regulation of gene transcription to stop. MYR=Myristylation signal; TM=Trans-membrane; SMBP=Small molecule binding protein; M=Mimic/blocker; TF=Transcription factor; NLS=Nuclear localization signal; SM=Small molecule; NES=Nuclear export signal.

FIG. 3—Schematic diagram illustrating the linear model of T-cell differentiation showing the expression markers associated with each cell type. APC—antigen-presenting cell; TCM—central memory T cell; TEFF—effector T cell; TEM—effector memory T cell; TN—naive T cell; TSCM—T memory stem cell.

FIG. 4—The tetracycline-responsive regulatory system for transcriptional transactivation

a) the tTA system: in this system the effector is a tetracycline-controlled transactivator (tTA) of transcription that consists of a chimeric construct of the Escherichia coli Tn10 tetR gene (purple) and the VP16 transactivation domain (orange). In the absence of the inducer, doxycycline (Dox), tTA dimers specifically bind to seven tandemly repeated 19-bp tetO sequences (tetO7), thereby activating transcription from a minimal promoter (TATA) and driving expression of the target transgene that encodes the gene of interest (target ORF). When bound to Dox, tTA undergoes a conformational change and cannot bind tetO sequences.

b) the reverse tTA (rtTA) system: in this system the tetR gene has been mutated so that it binds tetO sequences and activates transcription only in the presence of Dox.

FIG. 5—Graphs showing the proportion of Effector, Effector Memory (EM), Central Memory (CM) and Naïve cells following transduction (day 0) and 6 days after a 24 hour co-culture with CD19-expressing target cells (day 7). T cells were wither non-transduced (NT), transduced with a vector expressing the CAR only (HD37), or transduced with a vector expressing the CAR and the transcription factor FOXO1 (HD37-FOXO1). CD4+ and CD8+ subpopulations were analysed separately.

FIG. 6—Graphs showing the expression of CD27 and CD62L on CD4+ and CD8+ T cells 6 days after a 24 hour co-culture with CD19-expressing target cells. T cells were wither non-transduced (NT), transduced with a vector expressing the CAR only (HD37), transduced with a vector expressing the CAR and the transcription factor EOMES (HD37-EOMES) or transduced with a vector expressing the CAR and the transcription factor FOXO1 (HD37-FOXO1).

FIG. 7—Graphs showing the expression of CD62L on CD4+ and CD8+ T cells 6 days after a 24 hour co-culture with CD19-expressing target cells. T cells were wither non-transduced (NT), transduced with a vector expressing the CAR only (HD37), or transduced with a vector expressing the CAR and the transcription factors Runx3 and CBF beta (HD37-Runx3_CBFbeta).

FIG. 8—Graphs showing the proportion of Effector, Effector Memory (EM), Central Memory (CM) and Naïve cells following transduction (day 0) and 6 days after a 24 hour co-culture with CD19-expressing target cells (day 7). T cells were wither non-transduced (NT), transduced with a vector expressing the CAR only (HD37), transduced with a vector expressing the CAR and the transcription factor BACH2 (HD37-BACH2), or transduced with a vector expressing the CAR and a mutant version of the transcription factor BACH2 (HD37-BACH2_S520A). CD4+ and CD8+ subpopulations were analysed separately.

### SUMMARY OF ASPECTS OF THE INVENTION

The present inventors have developed a heterodimeric transcription system through which it is possible to turn transcription of a gene or a set of genes on or off using an external agent.

Thus in a first aspect the present invention provides a transcription system which comprises:
(a) a docking component which comprises a first binding domain; and
(b) a transcription control component which comprises a transcription factor and a second binding domain which binds the first binding domain of the docking component
wherein binding of the first and second binding domains is disrupted by the presence of an agent, such that in the absence of the agent the docking component and the transcription control component heterodimerize.

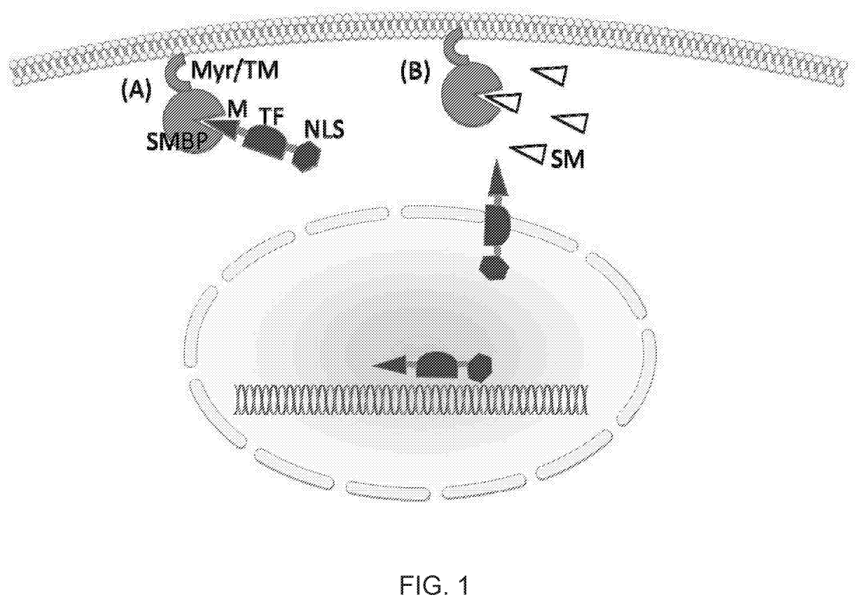

In a first embodiment of the first aspect of the invention, the docking component also comprises a membrane localisation domain; and the transcription component also comprises a nuclear localisation signal such that when the transcription system is expressed in a cell, in the absence of the agent the transcription component is held on the intracellular side of the plasma membrane; whereas in the presence of the agent the transcription component dissociates from the docking component and translocates to the nucleus where the transcription factor binds DNA and regulates the transcription of a gene (see FIG. 1).

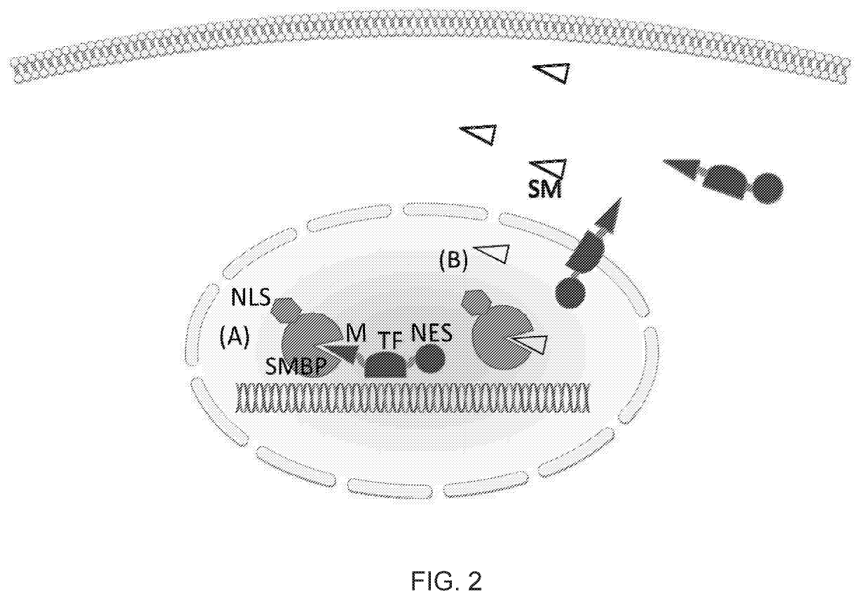

In a second embodiment of the first aspect of the invention, the docking component also comprises a nuclear localisation signal; and the transcription component also comprises a nuclear export signal such that when the transcription system is expressed in a cell, in the absence of the agent the transcription component is held in the nucleus where the transcription factor binds DNA and regulates the transcription of a gene; whereas in the presence of the agent the transcription component dissociates from the docking component and translocates to the cytoplasm (see FIG. 2).

The agent may be a small molecule which competitively inhibits the binding of the first and second binding domains.

For example, the first binding domain may comprise Tet Repressor Protein (TetR), and the second binding domain may comprise Transcription inducing peptide (TiP); or vice versa;
and the agent may be tetracycline, doxycycline or minocycline or an analogue thereof.

The first or second binding domain may comprise a single domain binder, such as: a nanobody, an affibody, a fibronectin artificial antibody scaffold, an anticalin, an affilin, a DARPin, a VNAR, an iBody, an affimer, a fynomer, a domain antibody (dAb), an abdurin/nanoantibody, a centyrin, an alphabody or a nanofitin.

The single domain binder may be or comprise a domain antibody (dAb).

In the transcription system of the first aspect of the invention either:
the first binding domain may comprise a single domain binder and the second binding domain may comprise a peptide which binds to the single domain binder, or
the second binding domain may comprise a single domain binder and the first binding domain may comprise a peptide which binds to the single domain binder,
which binding is competitively inhibited by the agent.

The agent may, for example, be tetracycline, doxycycline or minocycline.

The transcription factor may prevent or reduce T-cell differentiation and/or exhaustion when expressed in a T-cell.

For example, the transcription factor may promote central memory. The transcription factor is selected from the following group: EOMES, FOX01, Runx3, TCF1, LEF1 and ID3.

Alternatively, the transcription factor may promote effector memory. The transcription factor may be selected from the following group: T-bet, AP1, ID2, GATA3 and RORyt.

The transcription factor may be a central memory repressor. The transcription factor may be selected from the following group: BCL6 and BACH2.

The transcription factor may be an effector memory repressor, such as BLIMP-1.

The transcription factor may be or comprise Bach2 or a modified version of Bach2 which has reduced or removed capacity to be phosphorylated by ALK. A modified version of Bach2 may comprise a mutation at one or more of the following positions with reference to the amino acid sequence shown as SEQ ID No. 8: Ser-535, Ser-509, Ser-520.

The transcription factor may be FOXO1.

The transcription factor may be EOMES.

The transcription factor may comprise Runx3 and/or CBF beta.

In a second aspect the present invention provides a nucleic acid construct encoding a transcription system according to the first aspect of the invention, which comprises a first nucleic acid sequence encoding the docking component and a second nucleic acid sequence encoding the transcription control component.

The nucleic acid construct may have the following structure:
DC-coexpr-TCC; or
TCC-coexpr-DC
in which:
DC is a nucleic acid sequence encoding the docking component;
coexpr is a nucleic acid sequence enabling co-expression of the docking component and the transcription control component; and
TCC is a nucleic acid sequence encoding the transcription control component.

The nucleic acid construct may also comprise a third nucleic acid sequence encoding a chimeric antigen receptor.

In this respect, the nucleic acid construct may have one of the following structures:
CAR-coexprl -DC-coexpr2-TCC;
CAR-coexprl -TCC-coexpr2-DC;
DC-coexprl -TCC-coexpr2-CAR; or
TCC-coexprl -DC-coexp2-CAR
in which:
CAR is a nucleic acid sequence encoding a chimeric antigen receptor;
DC is a nucleic acid sequence encoding the docking component;
Coexpr1 and coexpr2, which may be the same or different, are nucleic acid sequences enabling co-expression of the docking component, the transcription control component and the chimeric antigen receptor; and
TCC is a nucleic acid sequence encoding the transcription control component.

In the above structures, coexpr, coexprl or coexpr2 may encode a sequence comprising a self-cleaving peptide.

In a third aspect, the present invention provides a kit of nucleic acid sequences which comprises a first nucleic acid sequence encoding a docking component as defined in the first aspect of the invention; and a second nucleic acid sequence encoding a transcription control component as defined in the first aspect of the invention.

The kit may also comprise a third nucleic acid sequence encoding a chimeric antigen receptor.

In a fourth aspect, the present invention provides a vector which comprises a nucleic acid construct according to the second aspect of the invention.

In a fifth aspect, the present invention provides a kit of vectors which comprises a first vector which comprises a first nucleic acid sequence encoding a docking component as defined in the first aspect of the invention; and a second vector which comprises a second nucleic acid sequence encoding a transcription control component as defined the first aspect of the invention.

The kit of vectors may also comprise a third vector which comprises a third nucleic acid sequence encoding a chimeric antigen receptor.

In a sixth aspect there is provided a cell which comprises a transcription system according to the first aspect of the invention.

The cell may express a chimeric antigen receptor.

In a seventh aspect the present invention provide a method for making a cell according to the sixth aspect of the invention, which comprises the step of introducing: a nucleic acid construct according to the second aspect of the invention, a kit of nucleic acid sequences according to the third aspect of the invention, a vector according to according to the fourth aspect of the invention, or a kit of vectors according to the fifth aspect of the invention, into a cell.

The cell may be from a sample isolated from a subject.

In a eighth aspect, there is provided a pharmaceutical composition comprising a plurality of cells according to the sixth aspect of the invention.

In a ninth aspect, there is provided a method for treating and/or preventing a disease, which comprises the step of administering a pharmaceutical composition according to the eighth aspect of the inevtnion to a subject.

The method may comprise the following steps:
(i) isolation of a cell-containing sample from a subject;
(ii) transduction or transfection of the cells with: a nucleic acid construct according to the second aspect of the invention, a kit of nucleic acid sequences according to the third aspect of the invention, a vector according to according to the fourth aspect of the invention, or a kit of vectors according to the fifth aspect of the invention; and
(iii) administering the cells from (ii) to the subject.

In a tenth aspect, there is provided a pharmaceutical composition according to the eighth aspect of the invention for use in treating and/or preventing a disease.

In an eleventh aspect there is provided the use of a cell according to the sixth aspect of the invention in the manufacture of a medicament for treating and/or preventing a disease.

In relation to the ninth, tenth and eleventh aspects of the invention, the disease may be a cancer.

In a twelfth aspect, there is provided amethod for regulating the transcription of a gene in a cell according to the sixth aspect of the invention, which comprises the step of administering the agent to the cell in vitro.

In a thirteenth aspect, there is provided a method for regulating the transcription of a gene in a cell according to the sixth aspect of the invention in vivo in a subject, which comprises the step of administering the agent to the subject.

The method according may comprise the following steps:
a) administration of a pharmaceutical composition according to the seventh aspect of the invention to a subject; and
b) administration of the agent to the subject
wherein a) and b) are administered in either order or simultaneously

In a fourteenth aspect the present invention provides a method for preventing or reducing T cell differentiation or exhaustion in a cell comprising a transcription system according to the first aspect of the invention comprising a transcription factor which prevents or reduces T-cell differentiation and/or exhaustion when expressed in a T-cell, which method comprises the step of administering the agent to a the cell in vitro.

In a fifteenth aspect, the present invention provides a method for preventing or reducing T cell differentiation or exhaustion in vivo in a subject in a cell comprising a transcription system according to the first aspect of the invention comprising a transcription factor which prevents or reduces T-cell differentiation and/or exhaustion when expressed in a T-cell, which method comprises the step of administering the agent to the subject.

The method may comprise the following steps:
a) administration of a pharmaceutical composition according to the seventh aspect of the invention to a subject, wherein the cells comprise a transcription system which prevents or reduces T-cell differentiation and/or exhaustion when expressed in a T-cell; and
b) administration of the agent to the subject
wherein a) and b) are administered in either order or simultaneously.

In a sixteenth aspect, the present invention provides a composition which comprises a plurality of cells according to the sixth aspect of the invention together with the agent which disrupts binding of the first and second binding domains.

The transcription system of the present invention uses a heterodimerization system, controllable by externally applied agent to control the location of a transcription factor within a cell and therefore its capacity to up- or down-regulate transcription of one or more target genes.

Where the transcription system utilises a natural transcription factor it is possible to control transcription of the target gene(s) associated with that transcription factor without engineering the target gene to comprise an artificial sequence element. This makes the system considerably more simple that the classical Tet-based systems which involve insertion of several TetO sequences upstream of the promoter.

Transcription may be turned on or off using the heterodimerization system of the invention depending on the intracellular location of the docking component (first and second embodiments of the first aspect of the invention, as shown in FIGS. 1 and 2 respectively). It is therefore possible to up- and down-regulate transcription using the same transcription factor, whether is it a suppressor or activator of transcription, by choosing the arrangement of domains in the docking and transcription control components.

It is also possible to select the heterodimerization system and the corresponding disrupting agent for use in the transcription system of the invention, meaning that agents can be chosen having desirable properties, such as being pharmacologically inert in mammalian cells, having a good volume of distribution and good cell penetration. The system of the invention is not limited to a particular hormone or antibiotic for its operation.

The present invention therefore provides a transcription system, controllable by an externally applied agent, which is simple, modular and highly flexible for the regulation of gene expression.

### DETAILED DESCRIPTION

Transcription System

The present invention provides a transcription system which comprises a docking component and a transcription control component. The docking component and transcriptional control component comprise dimerising binding domains, the interaction between which is disruptible by the presence of an agent.

Docking Component

The docking component acts as an anchor, tethering the transcription control component either in the cytoplasm, where is does not affect gene transcription; or in the nucleus, where it either up-regulates or down-regulates transcription of one or more target genes.

The docking component comprises a first heterodimerisation domain which interacts with a reciprocal domain on the transcription control component.

In the first embodiment of the invention, the docking component comprises a membrane localisation domain and (in the absence of agent) it causes the transcription control component to be located in the cytoplasm proximal to the plasma membrane.

In the second embodiment of the invention, the docking component comprises a nuclear localisation signal and (in the absence of agent) it causes the transcription control element to be located in the nucleus. When located in the nucleus, the transcription factor part of the transcription control element can up- or down-regulate transcription of one or more target genes.

Transcription Control Component

The transcription control component of the transcription system of the present invention comprises a transcription factor and a first heterodimerisation domain which interacts with a reciprocal domain on the docking component.

In the first embodiment of the invention, the transcription control component comprises a nuclear localisation signal so that when the transcription control component dissociates from the docking component in the presence of agent the transcription control component translocates to the nucleus where the transcription factor part of the transcription control element can up- or down-regulate transcription of one or more target genes

In the second embodiment of the invention, the transcription control component comprises a nuclear export signal so that when the transcription control component dissociates from the docking component in the presence of agent the transcription control component translocates out of the nucleus and transcription factor-mediated control of gene transcription is turned off.

Targeting Peptides

During the process of protein targeting in cells, proteins are directed to the correct intracellular location, i.e. an organelle, intracellular membrane, plasma membrane or the exterior of the cell via secretion; based on information contained in the protein itself.

The information may take the form of a targeting peptide, where it is a continuous stretch of amino acids; or a targeting patch, where it is comprises two or more stretches of sequence which are separate in the primary sequence of the polypeptide but brought together into a functional configuration after folding.

Targeting peptides or patches commonly comprise 3-70 amino acids. The sequence(s) directs the transport of a protein to a specific region in the cells, such as the nucleus, mitochondria, endoplasmic reticulum or plasma membrane.

Membrane Localisation Domain

In the first embodiment of the invention, the docking component comprises a membrane localisation domain. This may be any sequence which causes the docking component to be attached to or held in a position proximal to the plasma membrane.

It may be a sequence which causes the nascent polypeptide to be attached initially to the ER membrane. As membrane material “flows” from the ER to the Golgi and finally to the plasma membrane, the protein remain associated with the membrane at the end of the synthesis/translocation process.

The membrane localisation domain may, for example, comprise a transmembrane sequence, a stop transfer sequence, a GPI anchor or a myristoylation/prenylation/palmitoylation site.

Alternatively the membrane localisation domain may direct the docking component to a protein or other entity which is located at the cell membrane, for example by binding the membrane-proximal entity. The docking component may, for example, comprise a domain which binds a molecule which is involved in the immune synapse, such as TCR/CD3, CD4 or CD8.

Myristoylation is a lipidation modification where a myristoyl group, derived from myristic acid, is covalently attached by an amide bond to the alpha-amino group of an N-terminal glycine residue. Myristic acid is a 14-carbon saturated fatty acid also known as n-Tetradecanoic acid. The modification can be added either co-translationally or post-translationally. N-myristoyltransferase (NMT) catalyzes the myristic acid addition reaction in the cytoplasm of cells. Myristoylation causes membrane targeting of the protein to which it is attached, as the hydrophobic myristoyl group interacts with the phospholipids in the cell membrane.

The docking component of the present invention may comprise a sequence capable of being myristoylated by a NMT enzyme. The docking component of the present invention may comprise a myristoyl group when expressed in a cell.

The docking component may comprise a consensus sequence such as: NH2-G1 -X2-X3-X4-S5-X6-X7-X8 which is recognised by NMT enzymes.

Palmitoylation is the covalent attachment of fatty acids, such as palmitic acid, to cysteine and less frequently to serine and threonine residues of proteins. Palmitoylation enhances the hydrophobicity of proteins and can be used to induce membrane association. In contrast to prenylation and myristoylation, palmitoylation is usually reversible (because the bond between palmitic acid and protein is often a thioester bond). The reverse reaction is catalysed by palmitoyl protein thioesterases.

In signal transduction via G protein, palmitoylation of the α subunit, prenylation of the γ subunit, and myristoylation is involved in tethering the G protein to the inner surface of the plasma membrane so that the G protein can interact with its receptor.

The docking component of the present invention may comprise a sequence capable of being palmitoylated. The docking component of the present invention may comprise additional fatty acids when expressed in a cell which causes membrane localisation.

Prenylation (also known as isoprenylation or lipidation) is the addition of hydrophobic molecules to a protein or chemical compound. Prenyl groups (3-methyl-but-2-en-1-yl) facilitate attachment to cell membranes, similar to lipid anchors like the GPI anchor.

Protein prenylation involves the transfer of either a farnesyl or a geranyl-geranyl moiety to C-terminal cysteine(s) of the target protein. There are three enzymes that carry out prenylation in the cell, farnesyl transferase, Caax protease and geranylgeranyl transferase I.

The docking component of the present invention may comprise a sequence capable of being prenylated. The docking component of the present invention may comprise one or more prenyl groups when expressed in a cell which causes membrane localisation.

Nuclear Export Signal

A nuclear export signal (NES) is a short amino acid sequence of 4 hydrophobic residues in a protein that targets it for export from the cell nucleus to the cytoplasm through the nuclear pore complex using nuclear transport. It has the opposite effect of a nuclear localization signal, which targets a protein located in the cytoplasm for import to the nucleus. The NES is recognized and bound by exportins.

An NES often consists of several hydrophobic amino acids (often leucine) interspaced by 2-3 other amino acids. In silico analysis of known NESs found the most common spacing of the hydrophobic residues to be LxxxLxxLxL, where “L” is a hydrophobic residue (often leucine) and “x” is any other amino acid.

The NESdb database lists more than 200 nuclear export signals (Xu et al (2012) Mol Biol Cell 23:3677-3693).

Nuclear Localisation Signal

A nuclear localization signal or sequence (NLS) is an amino acid sequence that ‘tags’ a protein for import into the cell nucleus by nuclear transport. Typically, this signal consists of one or more short (for example 5 amino acid) sequences of positively charged amino acids, such as lysines or arginines, exposed on the protein surface. The NLS can be located anywhere on the polypeptide chain. Different nuclear localized proteins may share the same NLS.

Proteins gain entry into the nucleus through the nuclear envelope. The nuclear envelope consists of concentric membranes, the outer and the inner membrane. The inner and outer membranes connect at multiple sites, forming channels between the cytoplasm and the nucleoplasm. These channels are occupied by nuclear pore complexes (NPCs), complex multiprotein structures that mediate the transport across the nuclear membrane.

A protein translated with a NLS will bind strongly to importin (aka karyopherin), and, together, the complex will move through the nuclear pore.

Classical NLSs are either monopartite or bipartite. The first NLS to be discovered was the sequence PKKKRKV (SEQ ID No. 1) in the SV40 Large T-antigen (a monopartite NLS). The NLS of nucleoplasmin, KR[PAATKKAGQA]KKKK (SEQ ID No. 2), is the prototype of the ubiquitous bipartite signal: two clusters of basic amino acids, separated by a spacer of about 10 amino acids. Both signals are recognized by importin α.

Monopartite NLSs may have the consensus sequence K-K/R-X-K/R for example. It may be part of the downstream basic cluster of a bipartite NLS

Other examples of nuclear localisation signals include the eGFP fused NLSs of Nucleoplasmin (AVKRPAATKKAGQAKKKKLD—SEQ ID No. 3), EGL-13 (MSRRRKANPTKLSENAKKLAKEVEN—SEQ ID No. 4), c-Myc (PAAKRVKLD SEQ ID No. 5) and TUS-protein (KLKIKRPVK—SEQ ID No. 6).

There are many other types of “non-classical” NLSs, such as the acidic M9 domain of hnRNP A1, the sequence KIPIK in yeast transcription repressor Matα2, and the complex signals of U snRNPs. Most of these NLSs appear to be recognized directly by specific receptors of the importin β family without the intervention of an importin α-like protein.

Transcription Factor

The transcription control component of the transcription system of the invention comprises a transcription factor.

A transcription factor is a protein which controls the rate of transcription of genetic information from DNA to messenger RNA, by binding to a specific DNA sequence and regulate the expression of a gene which comprises or is adjacent to that sequence.

Transcription factors work by promoting (as an activator), or blocking (as a repressor) the recruitment of RNA polymerase.

In the first embodiment of the invention, the presence of agent causes the transcription control component (and therefore the transcription factor) to relocate to the nucleus. Where the transcription factor is an activator, the presence of agent in this system will therefore up-regulate transcription of the target gene. Where the transcription factor is a repressor, the presence of agent will down-regulate transcription of the target gene.

In the second embodiment of the invention, the presence of agent causes the transcription control component (and therefore the transcription factor) to leave the nucleus and relocate to the cytoplasm. Where the transcription factor is an activator, the presence of agent in this system will therefore down-regulate transcription of the target gene. Where the transcription factor is a repressor, the presence of agent will release the inhibition causing up-regulation of transcription of the target gene.

Transcription factors contain at least one DNA-binding domain (DBD), which attaches to either an enhancer or promoter region of DNA. Depending on the transcription factor, the transcription of the adjacent gene is either up- or down-regulated. Transcription factors also contain a trans-activating domain (TAD), which has binding sites for other proteins such as transcription coregulators.

Transcription factors use a variety of mechanisms for the regulation of gene expression, including stabilizing or blocking the binding of RNA polymerase to DNA, or catalyzing the acetylation or deacetylation of histone proteins. The transcription factor may have histone acetyltransferase (HAT) activity, which acetylates histone proteins, weakening the association of DNA with histones and making the DNA more accessible to transcription, thereby up-regulating transcription. Alternatively the transcription factor may have histone deacetylase (HDAC) activity, which deacetylates histone proteins, strengthening the association of DNA with histones and making the DNA less accessible to transcription, thereby down-regulating transcription. Another mechanism by which they may function is by recruiting coactivator or corepressor proteins to the transcription factor DNA complex.

There are two mechanistic classes of transcription factors, general transcription factors or upstream transcription factors.

General transcription factors are involved in the formation of a preinitiation complex. The most common are abbreviated as TFIIA, TFIIB, TFIID, TFIIE, TFIIF, and TFIIH. They are ubiquitous and interact with the core promoter region surrounding the transcription start site(s) of all class II genes.

Upstream transcription factors are proteins that bind upstream of the initiation site to stimulate or repress transcription. These are synonymous with specific transcription factors, because they vary considerably depending on what recognition sequences are present in the proximity of the gene.

Some examples of specific transcription factors are given in the table below:

| Factor | Structural type | Recognition sequence | Binds as |
|---|---|---|---|
| SP1 | Zinc finger | 5′-GGGCGG-3′ | Monomer |
| AP-1 | Basic zipper | 5′-TGA(G/C)TCA-3′ | Dimer |
| C/EBP | Basic zipper | 5′-ATTGCGCAAT-3′ | Dimer |
| Heat shock | Basic zipper | 5′-XGAAX-3′ | Trimer |
| factor | | | |
| ATF/CREB | Basic zipper | 5′-TGACGTCA-3′ | Dimer |
| c-Myc | Basic helix-loop- | 5′-CACGTG-3′ | Dimer |
| | helix | | |
| Oct-1 | Helix-turn-helix | 5′-ATGCAAAT-3′ | Monomer |
| NF-1 | Novel | 5′-TTGGCXXXXXGCCAA-3′ | Dimer |

Transcription factors are often classified based on the sequence similarity and hence the tertiary structure of their DNA-binding domains.

Transcription factors with basic domains include: leucine zipper factors (e.g. bZIP, c-Fos/c-Jun, CREB and Plant G-box binding factors); helix-loop-helix factors (e.g. Ubiquitous (class A) factors; myogenic transcription factors (MyoD); Achaete-Scute and Tal/Twist/Atonal/Hen); and helix-loop-helix/leucine zipper factors (e.g. bHLH-ZIP, c-Myc, NF-1 (A, B, C, X), RF-X (1, 2, 3, 4, 5, ANK) and bHSH).

Transcription factors with zinc-coordinating DNA-binding domains include the Cys4 zinc finger of nuclear receptor type such as steroid hormone receptors and thyroid hormone receptor-like factors; diverse Cys4 zinc fingers such as GATA-Factors; Cys2His2 zinc finger domains, such as ubiquitous factors, including TFIIIA, Sp1; developmental/cell cycle regulators, including Krüppel; large factors with NF-6B-like binding properties; Cys6 cysteine-zinc cluster and zinc fingers of alternating composition.

Transcription factors with helix-turn-helix domains include those with homeo domains; paired box; fork head/winged helix; heat shock factors; tryptophan clusters; and TEAs (transcriptional enhancer factor) domain such as TEAD1, TEAD2, TEAD3, TEAD4.

Finally there are beta-scaffold factors with minor groove contacts including the RHR (Rel homology region) class; STAT; p53; MADS box; beta-barrel alpha-helix transcription factors; TATA binding proteins; HMG-box; heteromeric CCAAT factors; Grainyhead; cold-shock domain factors; and Runt.

The transcription factor of the present invention may be constitutively active or conditionally active, i.e. requiring activation.

The transcription factor may be naturally occurring or artificial.

Repression of T-Cell Differentiation

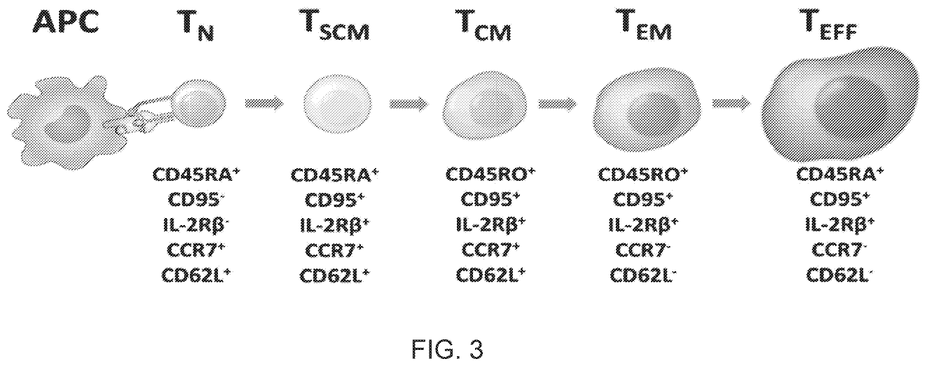

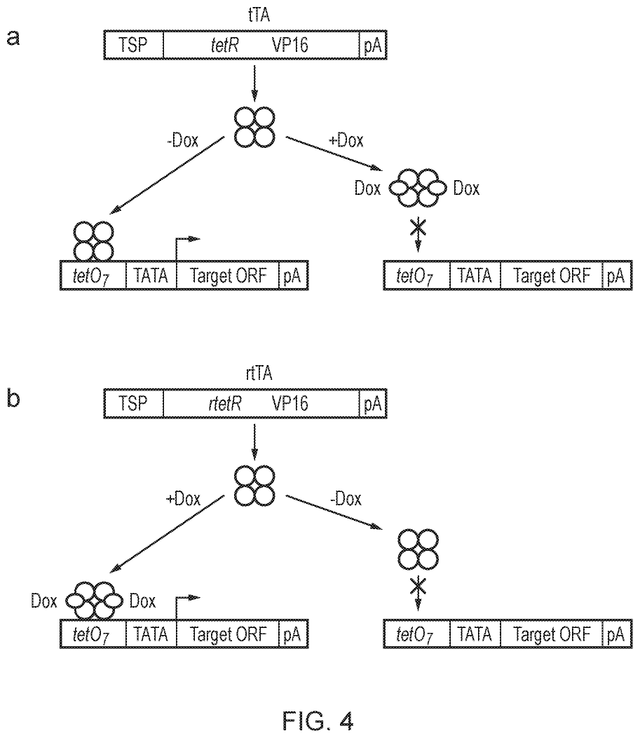

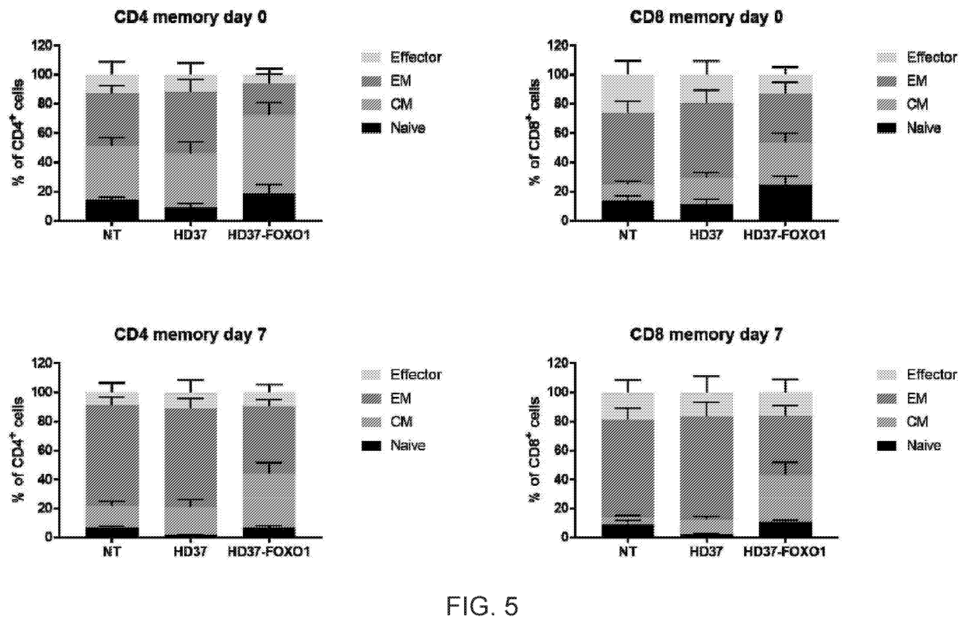

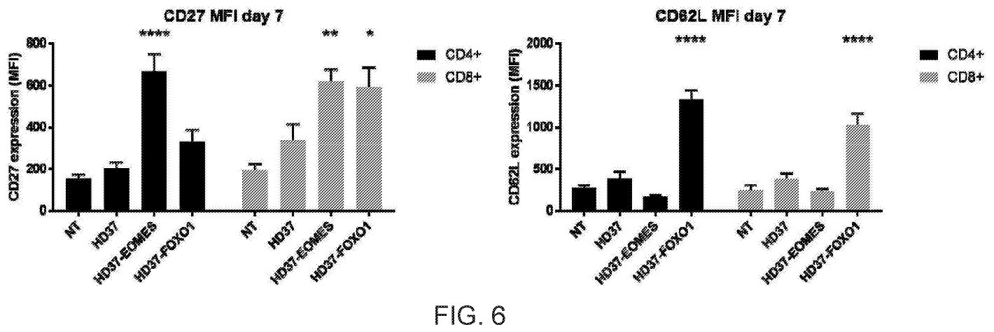

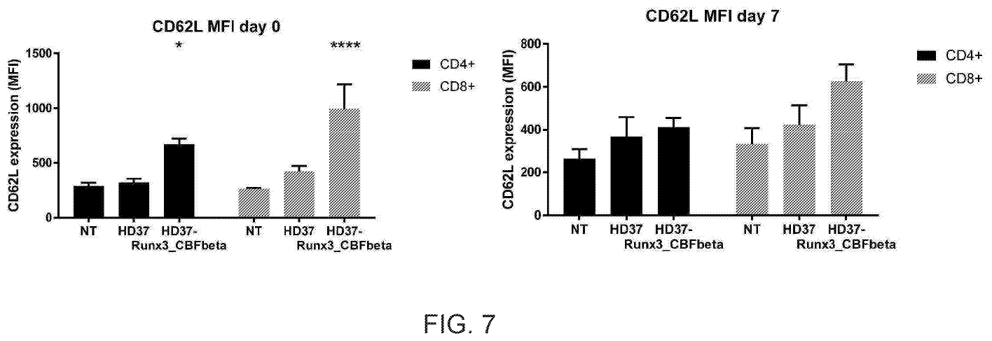

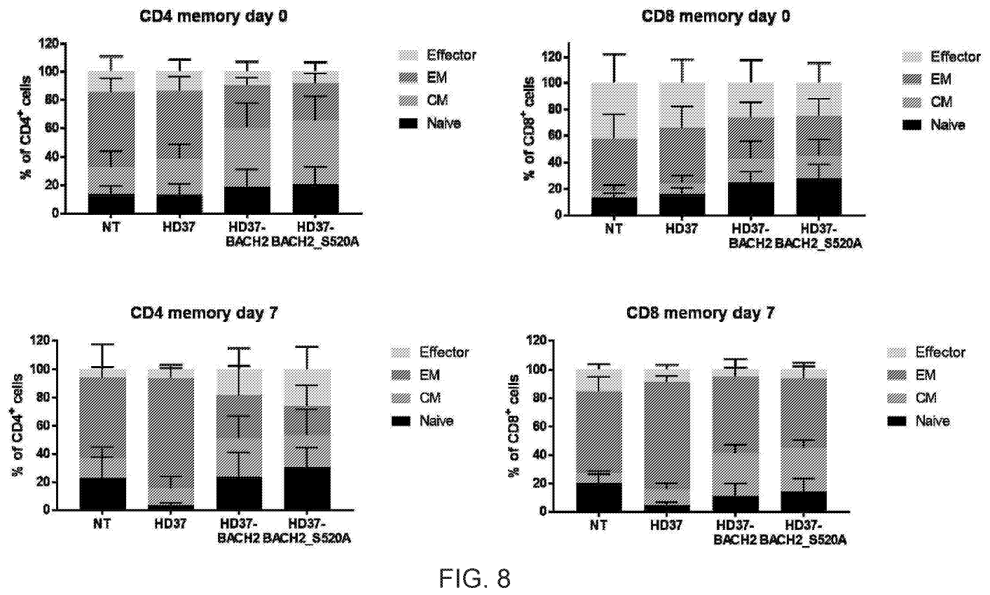

Following activation, T-cells differentiate into a variety of different T-cell subtypes, as shown in FIG. 3. In autologous immunotherapy approaches with T-cells, it is thought that T-cell persistence and engraftment in the subject is related to the proportion of nave, central memory and T-stem-cell memory T-cells administered to the subject.

The transcription system of the present invention may up- or down-regulate gene expression in a way which effectively increases the proportion of naïve, central memory and/or stem-cell memory T cells in the composition for administration to a patient.

In the first embodiment of the invention, the presence of the agent causes the transcription factor to translocate to the nucleus where it can exert its effect on the transcription of one or more target genes.

In connection with the first embodiment of the invention, the transcription factor may, for example be a central memory repressing transcription factor such as BCL6 or BACH2. Central memory repressors inhibit the differentiation of T cells to effector memory cells, so that they remain as one of the less differentiated T-cell subtypes, such a naïve and stem cell memory T-cells. They block or reduce the rate of differentiation of T cells through the various stages shown in FIG. 3, biasing the T-cell population towards a more nave phenotype.

Alternatively in connection with the first embodiment of the invention the transcription factor may be an effector memory repressing transcription factor such as BLIMP-1.

In the second embodiment of the invention, the presence of the agent causes the transcription factor to translocate to the cytoplasm so that it no longer affects the transcription of the target gene(s).

In connection with the second embodiment of the invention, the transcription factor may, for example be a central memory transcription factor such as EOMES, FOX01, Runx3, TCF1, LEF1 or ID3. Central memory transcription factors promote the differentiation of T cells to effector memory cells. Inhibition of a central memory transcription factor by the presence of the agent will block this function, meaning that the cells remain as one of the less differentiated T-cell subtypes, such a nave and stem cell memory T-cells.

Alternatively in connection with the second embodiment of the invention the transcription factor may be an effector memory transcription factors such as T-bet, AP1, ID2, GATA3 or RORγt.

BCL6

B-cell lymphoma protein (BCL6) is an evolutionarily conserved zinc finger transcription factor which contains an N-terminal POZ/BTB domain. BCL6 acts as a sequence-specific repressor of transcription, and has been shown to modulate the STAT-dependent Interleukin 4 (IL-4) responses of B cells. It interacts with several corepressor complexes to inhibit transcription.

The amino acid sequence of BCL6 is available from UniProt under accession No. P41182 and is shown as SEQ ID No. 7 below.

-BCL6
SEQ ID No. 7
MASPADSCIQFTRHASDVLLNLNRLRSRDILTDVVIVVSREQFRAHKTVL
MACSGLFYSIFTDQLKCNLSVINLDPEINPEGFCILLDFMYTSRLNLREG
NIMAVMATAMYLQMEHVVDTCRKFIKASEAEMVSAIKPPREEFLNSRMLM
PQDIMAYRGREVVENNLPLRSAPGCESRAFAPSLYSGLSTPPASYSMYSH
LPVSSLLFSDEEFRDVRMPVANPFPKERALPCDSARPVPGEYSRPTLEVS
PNVCHSNIYSPKETIPEEARSDMHYSVAEGLKPAAPSARNAPYFPCDKAS
KEEERPSSEDEIALHFEPPNAPLNRKGLVSPQSPQKSDCQPNSPTESCSS
KNACILQASGSPPAKSPTDPKACNWKKYKFIVLNSLNQNAKPEGPEQAEL
GRLSPRAYTAPPACQPPMEPENLDLQSPTKLSASGEDSTIPQASRLNNIV
NRSMTGSPRSSSESHSPLYMHPPKCTSCGSQSPQHAEMCLHTAGPTFPEE
MGETQSEYSDSSCENGAFFCNECDCRFSEEASLKRHTLQTHSDKPYKCDR
CQASFRYKGNLASHKTVHTGEKPYRCNICGAQFNRPANLKTHTRIHSGEK
PYKCETCGARFVQVAHLRAHVLIHTGEKPYPCEICGTRFRHLQTLKSHLR
IHTGEKPYHCEKCNLHFRHKSQLRLHLRQKHGAITNTKVQYRVSATDLPP
ELPKAC

BCL6 comprises six zinc fingers at the following amino acid positions: 518-541, 546-568, 574-596, 602-624, 630-652, 658-681.

BACH2

The broad complex and cap'n'collar homology (Bach)2 protein, also known as bric-a-brac and tramtrack, and is a 92 kDa transcriptional factor. Via a basic leucine zipper domain, it heterodimerizes with proteins of the musculoaponeurotic fibrosarcoma (Maf) family. The Bach2 gene locus resides in a Super Enhancer (SE), and regulates the expression of the SE-regulated genes. SEs are crucial for cell-lineage gene expression. In T-cells, the majority of SE-regulated genes are cytokines and cytokine receptor genes. Bach2 is a predominant gene associated with SE in all T-cell lineages.

The Bach2 protein consists of 72 phosphorylation sites. Of those sites, Ser-335 consists of the consensus sequence of Akt targets (RXRXX(S/T)X). Eleven sites (Ser-260, Ser-314, Thr-318, Thr-321, Ser-336, Ser-408, Thr-442, Ser-509, Ser-535, Ser-547, and Ser-718) bear the consensus sequence of mTOR targets (proline at +1 position). Substitution of Ser-535 and Ser-509 to Ala increases the nuclear localisation of Bach2, and augments the downregulation of its target genes.

The site Ser-520 has been identified as an Akt substrate for phosphorylation. Substitution of Ser-520 to Ala also increases the repressor capacity of Bach2. eGFP fusion to the WT or mutated Bach2 revealed an augmented nuclear localisation of S520A Bach2. The phosphorylation of Bach2 upon T-cell activation leads to Bach2 sequestration in the cytoplasm. Mutations at the phosphorylation site render Bach2 resistant to such sequestration, and thus its localisation to the nucleus is increased.

The transcription control component may comprise a variant of Bach2 which has increased nuclear localisation compared to the wild type protein. The variant may have a mutation at Ser-535, Ser-520 or Ser-509 with reference to the sequence shown as SEQ ID No. 8. The mutation may be a substitution, such as a Ser to Ala substitution.

Bach2 binds on the consensus motif (5′-TGA(C/G)TCAGC-3′), which is part of the motif (5′-TGA(C/G)TCA-3′) recognised by the AP-1 family. AP-1 family of transcription factors is involved in inducing the expression of genes downstream of TCR activation. The AP-1 transcription factor family includes c-Jun, JunB and c-Fos. AP-1 factors are phosphorylated upon TCR activation, and subsequently regulate genes involved in effector differentiation. Bach2 represses the activation of those genes, by competing with AP-1 for binding on overlapping motifs.

The expression of Bach2 mRNA is high in nave CD8 T-cells, and is gradually downregulated in central memory (CD62L+KLRG1−), effector (CD62L−KLRG1−) and terminally differentiated effector (CD62L−KLRG1+) cells. Deficiency of Bach2 leads to terminally differentiated T-cells, and increases apoptosis.

The amino acid sequence of Bac2 is available from UniProt under accession No. Q9BYV9 and is shown as SEQ ID No. 8 below.

-Bach-2 wild type
SEQ ID No. 8
MSVDEKPDSPMYVYESTVHCTNILLGLNDQRKKDILCDVTLIVERKEFRA
HRAVLAACSEYFWQALVGQTKNDLVVSLPEEVTARGFGPLLQFAYTAKLL
LSRENIREVIRCAEFLRMHNLEDSCFSFLQTQLLNSEDGLFVCRKDAACQ
RPHEDCENSAGEEEDEEEETMDSETAKMACPRDQMLPEPISFEAAAIPVA
EKEEALLPEPDVPTDTKESSEKDALTQYPRYKKYQLACTKNVYNASSHST
SGFASTFREDNSSNSLKPGLARGQIKSEPPSEENEEESITLCLSGDEPDA
KDRAGDVEMDRKQPSPAPTPTAPAGAACLERSRSVASPSCLRSLFSITKS
VELSGLPSTSQQHFARSPACPFDKGITQGDLKTDYTPFTGNYGQPHVGQK
EVSNFTMGSPLRGPGLEALCKQEGELDRRSVIFSSSACDQVSTSVHSYSG
VSSLDKDLSEPVPKGLWVGAGQSLPSSQAYSHGGLMADHLPGRMRPNTSC
PVPIKVCPRSPPLETRTRTSSSCSSYSYAEDGSGGSPCSLPLCEFSSSPC
SQGARFLATEHQEPGLMGDGMYNQVRPQIKCEQSYGTNSSDESGSFSEAD
SESCPVQDRGQEVKLPFPVDQITDLPRNDFQMMIKMHKLTSEQLEFIHDV
RRRSKNRIAAQRCRKRKLDCIQNLECEIRKLVCEKEKLLSERNQLKACMG
ELLDNFSCLSQEVCRDIQSPEQIQALHRYCPVLRPMDLPTASSINPAPLG
AEQNIAASQCAVGENVPCCLEPGAAPPGPPWAPSNTSENCTSGRRLEGTD
PGTFSERGPPLEPRSQTVTVDFCQEMTDKCTTDEQPRKDYT

A mutant Bach2 sequence which has an S to A substitution at position 520 is shown as SEQ ID No. 9. The S520A substitution is in bold and underlined.

-S520A Bach2 mutant (insensitive to AKT)
SEQ ID No. 9
MSVDEKPDSPMYVYESTVHCTNILLGLNDQRKKDILCDVTLIVERKEFRA
HRAVLAACSEYFWQALVGQTKNDLVVSLPEEVTARGFGPLLQFAYTAKLL
LSRENIREVIRCAEFLRMHNLEDSCFSFLQTQLLNSEDGLFVCRKDAACQ
RPHEDCENSAGEEEDEEEETMDSETAKMACPRDQMLPEPISFEAAAIPVA
EKEEALLPEPDVPTDTKESSEKDALTQYPRYKKYQLACTKNVYNASSHST
SGFASTFREDNSSNSLKPGLARGQIKSEPPSEENEEESITLCLSGDEPDA
KDRAGDVEMDRKQPSPAPTPTAPAGAACLERSRSVASPSCLRSLFSITKS
VELSGLPSTSQQHFARSPACPFDKGITQGDLKTDYTPFTGNYGQPHVGQK
EVSNFTMGSPLRGPGLEALCKQEGELDRRSVIFSSSACDQVSTSVHSYSG
VSSLDKDLSEPVPKGLWVGAGQSLPSSQAYSHGGLMADHLPGRMRPNTSC
PVPIKVCPRSPPLETRTRTSASCSSYSYAEDGSGGSPCSLPLCEFSSSPC
SQGARFLATEHQEPGLMGDGMYNQVRPQIKCEQSYGINSSDESGSFSEAD
SESCPVQDRGQEVKLPFPVDQITDLPRNDFQMMIKMHKLTSEQLEFIHDV
RRRSKNRIAAQRCRKRKLDCIQNLECEIRKLVCEKEKLLSERNQLKACMG
ELLDNFSCLSQEVCRDIQSPEQIQALHRYCPVLRPMDLPTASSINPAPLG
AEQNIAASQCAVGENVPCCLEPGAAPPGPPWAPSNTSENCTSGRRLEGTD
PGTFSERGPPLEPRSQTVTVDFCQEMTDKCTTDEQPRKDYT

BLIMP-1

B-lymphocyte-induced maturation protein 1 (BLIMP1) acts as a repressor of beta-interferon (β-IFN) gene expression. The protein binds specifically to the PRDI (positive regulatory domain I element) of the β-IFN gene promoter.

The increased expression of the Blimp-1 protein in B lymphocytes, T lymphocytes, NK cell and other immune system cells leads to an immune response through proliferation and differentiation of antibody secreting plasma cells. Blimp-1 is also considered a ‘master regulator’ of hematopoietic stem cells.

BLIMP-1 is involved in controlling the terminal differentiation of antibody-secreting cells (ASCs) and has an important role in maintaining the homeostasis of effector T cells.

The amino acid sequence of BLIMP-1 is available from UniProt under accession No. O75626 and is shown as SEQ ID No. 10 below.

-BLIMP-1
SEQ ID No. 10
MLDICLEKRVGTTLAAPKCNSSTVRFQGLAEGTKGTMKMDMEDADMTLWT
EAEFEEKCTYIVNDHPWDSGADGGTSVQAEASLPRNLLFKYATNSEEVIG
VMSKEYIPKGTRFGPLIGEIYTNDTVPKNANRKYFWRIYSRGELHHFIDG
FNEEKSNWMRYVNPAHSPREQNLAACQNGMNIYFYTIKPIPANQELLVWY
CRDFAERLHYPYPGELTMMNLTQTQSSLKQPSTEKNELCPKNVPKREYSV
KEILKLDSNPSKGKDLYRSNISPLTSEKDLDDFRRRGSPEMPFYPRVVYP
IRAPLPEDFLKASLAYGIERPTYITRSPIPSSTTPSPSARSSPDQSLKSS
SPHSSPGNIVSPVGPGSQEHRDSYAYLNASYGTEGLGSYPGYAPLPHLPP
AFIPSYNAHYPKFLLPPYGMNCNGLSAVSSMNGINNFGLFPRLCPVYSNL
LGGGSLPHPMLNPTSLPSSLPSDGARRLLQPEHPREVLVPAPHSAFSFTG
AAASMKDKACSPTSGSPTAGTAATAEHVVQPKATSAAMAAPSSDEAMNLI
KNKRNMTGYKTLPYPLKKQNGKIKYECNVCAKTFGQLSNLKVHLRVHSGE
RPFKCQTCNKGFTQLAHLQKHYLVHTGEKPHECQVCHKRFSSTSNLKTHL
RLHSGEKPYQCKVCPAKFTQFVHLKLHKRLHTRERPHKCSQCHKNYIHLC
SLKVHLKGNCAAAPAPGLPLEDLTRINEEIEKFDISDNADRLEDVEDDIS
VISVVEKEILAVVRKEKEETGLKVSLQRNMGNGLLSSGCSLYESSDLPLM
KLPPSNPLPLVPVKVKQETVEPMDP

EOMES

Eomesodermin (Eomes), also known as T-box brain protein 2 (Tbr2), is a protein that in humans is encoded by the EOMES gene. T-box genes encode transcription factors. Eomes has a role in immune response and is highly expressed in CD8+ T cells but not CD4+ T cells.

The amino acid sequence of Eomes is available from UniProt under accession No. O95936 and is shown as SEQ ID No. 11 below.

-EOMES
SEQ ID No. 11
MQLGEQLLVS SVNLPGAHFY PLESARGGSG GSAGHLPSAA
PSPQKLDLDK ASKKFSGSLS CEAVSGEPAA ASAGAPAAML
SDTDAGDAFA SAAAVAKPGP PDGRKGSPCG EEELPSAAAA
AAAAAAAAAA TARYSMDSLS SERYYLQSPG PQGSELAAPC
SLFPYQAAAG APHGPVYPAP NGARYPYGSM LPPGGFPAAV
CPPGRAQFGP GAGAGSGAGG SSGGGGGPGT YQYSQGAPLY
GPYPGAAAAG SCGGLGGLGV PGSGFRAHVY LCNRPLWLKF
HRHQTEMIIT KQGRRMFPFL SFNINGLNPT AHYNVFVEVV
LADPNHWRFQ GGKWVTCGKA DNNMQGNKMY VHPESPNTGS
HWMRQEISFG KLKLTNNKGA NNNNTQMIVL QSLHKYQPRL
HIVEVTEDGV EDLNEPSKTQ TFTFSETQFI AVTAYQNTDI
TQLKIDHNPF AKGFRDNYDS SHQIVPGGRY GVQSFFPEPF
VNTLPQARYY NGERTVPQTN GLLSPQQSEE VANPPQRWLV
TPVQQPGTNK LDISSYESEY TSSTLLPYGI KSLPLQTSHA
LGYYPDPTFP AMAGWGGRGS YQRKMAAGLP WTSRTSPTVF
SEDQLSKEKV KEEIGSSWIE TPPSIKSLDS NDSGVYTSAC
KRRRLSPSNS SNENSPSIKC EDINAEEYSK DTSKGMGGYY
AFYTTP

FOX01

Forkhead box protein O1 (FOXO1) also known as forkhead in rhabdomyosarcoma is a protein that in humans is encoded by the FOXO1 gene. FOXO1 is a transcription factor that plays important roles in regulation of gluconeogenesis and glycogenolysis by insulin signaling, and is also central to the decision for a preadipocyte to commit to adipogenesis.

The amino acid sequence of FOX01 is available from UniProt under accession No. O12778 and is shown as SEQ ID No. 12 below.

-FOX01
SEQ ID No. 12
MAEAPQVVEI DPDFEPLPRP RSCTWPLPRP EFSQSNSATS
SPAPSGSAAA NPDAAAGLPS ASAAAVSADF MSNLSLLEES
EDFPQAPGSV AAAVAAAAAA AATGGLCGDF QGPEAGCLHP
APPQPPPPGP LSQHPPVPPA AAGPLAGQPR KSSSSRRNAW
GNLSYADLIT KAIESSAEKR LTLSQIYEWM VKSVPYFKDK
GDSNSSAGWK NSIRHNLSLH SKFIRVQNEG TGKSSWWMLN
PEGGKSGKSP RRRAASMDNN SKFAKSRSRA AKKKASLQSG
QEGAGDSPGS QFSKWPASPG SHSNDDFDNW STFRPRTSSN
ASTISGRLSP IMTEQDDLGE GDVHSMVYPP SAAKMASTLP
SLSEISNPEN MENLLDNLNL LSSPTSLTVS TQSSPGTMMQ
QTPCYSFAPP NTSLNSPSPN YQKYTYGQSS MSPLPQMPIQ
TLQDNKSSYG GMSQYNCAPG LLKELLTSDS PPHNDIMTPV
DPGVAQPNSR VLGQNVMMGP NSVMSTYGSQ ASHNKMMNPS
SHTHPGHAQQ TSAVNGRPLP HTVSTMPHTS GMNRLTQVKT
PVQVPLPHPM QMSALGGYSS VSSCNGYGRM GLLHQEKLPS
DLDGMFIERL DCDMESIIRN DLMDGDTLDF NFDNVLPNQS
FPHSVKTTTH SWVSG

RUNX3

Runt-related transcription factor 3 (Runx3) is a member of the runt domain-containing family of transcription factors. A heterodimer of this protein and a beta subunit forms a complex that binds to the core DNA sequence 5′-YGYGGT-3′ found in a number of enhancers and promoters, which can either activate or suppress transcription.

The amino acid sequence of RUNX3 is available from UniProt under accession No. O13761 and is shown as SEQ ID No. 13 below.

SEQ ID No. 13-RUNX3
MRIPVDPSTS RRFTPPSPAF PCGGGGGKMG ENSGALSAQA
AVGPGGRARP EVRSMVDVLA DHAGELVRTD SPNFLCSVLP
SHWRCNKTLP VAFKVVALGD VPDGTVVTVM AGNDENYSAE
LRNASAVMKN QVARFNDLRF VGRSGRGKSF TLTITVFTNP
TQVATYHRAI KVTVDGPREP RRHRQKLEDQ TKPFPDRFGD
LERLRMRVTP STPSPRGSLS TTSHFSSQPQ TPIQGTSELN
PFSDPRQFDR SFPTLPTLTE SRFPDPRMHY PGAMSAAFPY
SATPSGTSIS SLSVAGMPAT SRFHHTYLPP PYPGAPQNQS
GPFQANPSPY HLYYGTSSGS YQFSMVAGSS SGGDRSPTRM
LASCTSSAAS VAAGNLMNPS LGGQSDGVEA DGSHSNSPTA
LSTPGRMDEA VWRPY

TCF1

TCF-1, also known as HNF-1α, is a transcription factor expressed in organs of endoderm origin, including liver, kidneys, pancreas, intestines, stomach, spleen, thymus, testis, and keratinocytes and melanocytes in human skin. It has been shown to affect intestinal epithelial cell growth and cell lineages differentiation.

The amino acid sequence of TCF-1 is available from UniProt under accession No. P20823 and is shown as SEQ ID No. 14 below.

SEQ ID No. 14-TCF1
MVSKLSQLQT ELLAALLESG LSKEALIQAL GEPGPYLLAG
EGPLDKGESC GGGRGELAEL PNGLGETRGS EDETDDDGED
FTPPILKELE NLSPEEAAHQ KAVVETLLQE DPWRVAKMVK
SYLQQHNIPQ REVVDTTGLN QSHLSQHLNK GTPMKTQKRA
ALYTWYVRKQ REVAQQFTHA GQGGLIEEPT GDELPTKKGR
RNRFKWGPAS QQILFQAYER QKNPSKEERE TLVEECNRAE
CIQRGVSPSQ AQGLGSNLVT EVRVYNWFAN RRKEEAFRHK
LAMDTYSGPP PGPGPGPALP AHSSPGLPPP ALSPSKVHGV
RYGQPATSET AEVPSSSGGP LVTVSTPLHQ VSPTGLEPSH
SLLSTEAKLV SAAGGPLPPV STLTALHSLE QTSPGLNQQP
QNLIMASLPG VMTIGPGEPA SLGPTFTNTG ASTLVIGLAS
TQAQSVPVIN SMGSSLTTLQ PVQFSQPLHP SYQQPLMPPV
QSHVTQSPFM ATMAQLQSPH ALYSHKPEVA QYTHTGLLPQ
TMLITDTTNL SALASLTPTK QVFTSDTEAS SESGLHTPAS
QATTLHVPSQ DPAGIQHLQP AHRLSASPTV SSSSLVLYQS
SDSSNGQSHL LPSNHSVIET FISTQMASSS Q

LEF1

Lymphoid enhancer-binding factor-1 (LEF1) is a 48-kD nuclear protein that is expressed in pre-B and T cells. It binds to a functionally important site in the T-cell receptor-alpha (TCRA) enhancer and confers maximal enhancer activity. LEF1 belongs to a family of regulatory proteins that share homology with high mobility group protein-1 (HMG1).

The amino acid sequence of LEF1 is available from UniProt under accession No. Q9UJU2 and is shown as SEQ ID No. 15 below.

SEQ ID No. 15-LEF1
MPQLSGGGGG GGGDPELCAT DEMIPFKDEG DPQKEKIFAE
ISHPEEEGDL ADIKSSLVNE SEIIPASNGH EVARQAQTSQ
EPYHDKAREH PDDGKHPDGG LYNKGPSYSS YSGYIMMPNM
NNDPYMSNGS LSPPIPRTSN KVPVVQPSHA VHPLTPLITY
SDEHFSPGSH PSHIPSDVNS KQGMSRHPPA PDIPTFYPLS
PGGVGQITPP LGWQGQPVYP ITGGFRQPYP SSLSVDTSMS
RFSHHMIPGP PGPHTTGIPH PAIVTPQVKQ EHPHTDSDLM
HVKPQHEQRK EQEPKRPHIK KPLNAFMLYM KEMRANVVAE
CTLKESAAIN QILGRRWHAL SREEQAKYYE LARKERQLHM
QLYPGWSARD NYGKKKKRKR EKLQESASGT GPRMTAAYI

ID3

DNA-binding protein inhibitor ID-3 is a member of the ID family of helix-loop-helix (HLH) proteins which lack a basic DNA-binding domain and inhibit transcription through formation of nonfunctional dimers that are incapable of binding to DNA.

The amino acid sequence of ID3 is available from UniProt under accession No. Q02535 and is shown as SEQ ID No. 16 below.

SEQ ID No. 16-ID3
MKALSPVRGC YEAVCCLSER SLAIARGRGK GPAAEEPLSL
LDDMNHCYSR LRELVPGVPR GTQLSQVEIL QRVIDYILDL
QVVLAEPAPG PPDGPHLPIQ TAELTPELVI SNDKRSFCH

T-BET

T-bet, or T-box transcription factor TBX21 is encoded by the TBX21 gene, a member of a phylogenetically conserved family of genes that share a common DNA-binding domain, the T-box. T-box genes encode transcription factors involved in the regulation of developmental processes. This gene is the human ortholog of mouse Tbx21/Tbet gene. Studies in mouse show that Tbx21 protein is a Th1 cell-specific transcription factor that controls the expression of the hallmark Th1 cytokine, interferon-gamma (IFNG). Expression of the human ortholog also correlates with IFNG expression in Th1 and natural killer cells, suggesting a role for this gene in initiating Th1 lineage development from naive Th precursor cells.

The amino acid sequence of T-bet is available from UniProt under accession No. Q9UL17 and is shown as SEQ ID No. 17 below.

SEQ ID No. 17-T-bet
MGIVEPGCGD MLTGTEPMPG SDEGRAPGAD PQHRYFYPEP
GAQDADERRG GGSLGSPYPG GALVPAPPSR FLGAYAYPPR
PQAAGFPGAG ESFPPPADAE GYQPGEGYAA PDPRAGLYPG
PREDYALPAG LEVSGKLRVA LNNHLLWSKF NQHQTEMIIT
KQGRRMFPFL SFTVAGLEPT SHYRMFVDVV LVDQHHWRYQ
SGKWVQCGKA EGSMPGNRLY VHPDSPNTGA HWMRQEVSFG
KLKLTNNKGA SNNVTQMIVL QSLHKYQPRL HIVEVNDGEP
EAACNASNTH IFTFQETQFI AVTAYQNAEI TQLKIDNNPF
AKGFRENFES MYTSVDTSIP SPPGPNCQFL GGDHYSPLLP
NQYPVPSRFY PDLPGQAKDV VPQAYWLGAP RDHSYEAEFR
AVSMKPAFLP SAPGPTMSYY RGQEVLAPGA GWPVAPQYPP
KMGPASWFRP MRTLPMEPGP GGSEGRGPED QGPPLVWTEI
APIRPESSDS GLGEGDSKRR RVSPYPSSGD SSSPAGAPSP
FDKEAEGQFY NYFPN

AP1

Activator protein 1 (AP-1) is a transcription factor that regulates gene expression in response to a variety of stimuli, including cytokines, growth factors, stress, and bacterial and viral infections. AP-1 controls a number of cellular processes including differentiation, proliferation, and apoptosis. The structure of AP-1 is a heterodimer composed of proteins belonging to the c-Fos, c-Jun, ATF and JDP families.

The amino acid sequence of AP1 is available from UniProt under accession No. P05412 and is shown as SEQ ID No. 18 below.

SEQ ID No. 18-AP1
MTAKMETTFY DDALNASFLP SESGPYGYSN PKILKQSMTL
NLADPVGSLK PHLRAKNSDL LTSPDVGLLK LASPELERLI
IQSSNGHITT TPTPTQFLCP KNVTDEQEGF AEGFVRALAE
LHSQNTLPSV TSAAQPVNGA GMVAPAVASV AGGSGSGGFS
ASLHSEPPVY ANLSNFNPGA LSSGGGAPSY GAAGLAFPAQ
PQQQQQPPHH LPQQMPVQHP RLQALKEEPQ TVPEMPGETP
PLSPIDMESQ ERIKAERKRM RNRIAASKCR KRKLERIARL
EEKVKTLKAQ NSELASTANM LREQVAQLKQ KVMNHVNSGC
QLMLTQQLQT F

ID2

DNA-binding protein inhibitor ID-2 belongs to the inhibitor of DNA binding (ID) family, members of which are transcriptional regulators that contain a helix-loop-helix (HLH) domain but not a basic domain. Members of the ID family inhibit the functions of basic helix-loop-helix transcription factors in a dominant-negative manner by suppressing their heterodimerization partners through the HLH domains. This protein may play a role in negatively regulating cell differentiation.

The amino acid sequence of ID2 is available from UniProt under accession No. Q02363 and is shown as SEQ ID No. 19 below.

SEQ ID No. 19-ID2
MKAFSPVRSV RKNSLSDHSL GISRSKTPVD DPMSLLYNMN
DCYSKLKELV PSIPQNKKVS KMEILQHVID YILDLQIALD
SHPTIVSLHH QRPGQNQASR TPLTTLNTDI SILSLQASEF
PSELMSNDSK ALCG

GATA3

Trans-acting T-cell-specific transcription factor GATA-3 belongs to the GATA family of transcription factors. It regulates luminal epithelial cell differentiation in the mammary gland. The protein contains two GATA-type zinc fingers and is an important regulator of T cell development. GATA-3 has been shown to promote the secretion of IL-4, IL-5, and IL-13 from Th2 cells, and induce the differentiation of Th0 cells towards this Th2 cell subtype while suppressing their differentiation towards Th1 cells.

The amino acid sequence of GATA3 is available from UniProt under accession No. P23771 and is shown as SEQ ID No. 20 below.

SEQ ID No. 20-GATA3
MEVTADQPRW VSHHHPAVLN GQHPDTHHPG LSHSYMDAAQ
YPLPEEVDVL FNIDGQGNHV PPYYGNSVRA TVQRYPPTHH
GSQVCRPPLL HGSLPWLDGG KALGSHHTAS PWNLSPFSKT
SIHHGSPGPL SVYPPASSSS LSGGHASPHL FTFPPTPPKD
VSPDPSLSTP GSAGSARQDE KECLKYQVPL PDSMKLESSH
SRGSMTALGG ASSSTHHPIT TYPPYVPEYS SGLFPPSSLL
GGSPTGFGCK SRPKARSSTG RECVNCGATS TPLWRRDGTG
HYLCNACGLY HKMNGQNRPL IKPKRRLSAA RRAGTSCANC
QTTTTTLWRR NANGDPVCNA CGLYYKLHNI NRPLTMKKEG
IQTRNRKMSS KSKKCKKVHD SLEDFPKNSS FNPAALSRHM
SSLSHISPFS HSSHMLTTPT PMHPPSSLSF GPHHPSSMVT
AMG

RORγt

RAR-related orphan receptor gamma (RORγ) is a member of the nuclear receptor family of transcription factors, which has two isoforms: RORy and RORyt. The tissue distribution of the second isoform, RORγt, appears to be highly restricted to the thymus where it is expressed exclusively in immature CD4+/CD8+ thymocytes and in lymphoid tissue inducer (LTi) cells. RORγt is essential for lymphoid organogenesis, in particular lymph nodes and Peyer's patches, but not the spleen. It plays an important regulatory role in thymopoiesis, by reducing apoptosis of thymocytes and promoting thymocyte differentiation into pro-inflammatory T helper 17 (Th17) cells. It also plays a role in inhibiting apoptosis of undifferentiated T cells and promoting their differentiation into Th17 cells, possibly by down regulating the expression of Fas ligand and IL2, respectively.

The amino acid sequence of RORγt is available from UniProt under accession No. P51449 and is shown as SEQ ID No. 21 below.

SEQ ID No. 21-RORγt
MDRAPQRQHR ASRELLAAKK THTSQIEVIP CKICGDKSSG
IHYGVITCEG CKGFFRRSQR CNAAYSCTRQ QNCPIDRTSR
NRCQHCRLQK CLALGMSRDA VKFGRMSKKQ RDSLHAEVQK
QLQQRQQQQQ EPVVKTPPAG AQGADTLTYT LGLPDGQLPL
GSSPDLPEAS ACPPGLLKAS GSGPSYSNNL AKAGLNGASC
HLEYSPERGK AEGRESFYST GSQLTPDRCG LRFEEHRHPG
LGELGQGPDS YGSPSFRSTP EAPYASLTEI EHLVQSVCKS
YRETCQLRLE DLLRQRSNIF SREEVTGYQR KSMWEMWERC
AHHLTEAIQY VVEFAKRLSG FMELCONDQI VLLKAGAMEV
VLVRMCRAYN ADNRTVFFEG KYGGMELFRA LGCSELISSI
FDFSHSLSAL HFSEDEIALY TALVLINAHR PGLQEKRKVE
QLQYNLELAF HHHLCKTHRQ SILAKLPPKG KLRSLCSQHV
ERLQIFQHLH PIVVQAAFPP LYKELFSTET ESPVGLSK

CBF BETA

Core-binding factor subunit beta (CBF beta) is the beta subunit of a heterodimeric core-binding transcription factor belonging to the PEBP2/CBF transcription factor family which master-regulates a host of genes specific to hematopoiesis (e.g., RUNX1) and osteogenesis (e.g., RUNX2). The beta subunit is a non-DNA binding regulatory subunit; it allosterically enhances DNA binding by the alpha subunit as the complex binds to the core site of various enhancers and promoters, including murine leukemia virus, polyomavirus enhancer, T-cell receptor enhancers and GM-CSF promoters. Alternative splicing generates two mRNA variants, each encoding a distinct carboxyl terminus.

The amino acid sequence of CBF beta is available from UniProt under accession No. Q13951 and is shown below as SEQ ID No. 22.

SEQ ID No. 22-CBF beta
MPRVVPDQRSKFENEEFFRKLSRECEIKYTGFRDRPHEERQARFQNACRD
GRSEIAFVATGTNLSLQFFPASWQGEQRQTPSREYVDLEREAGKVYLKAP
MILNGVCVIWKGWIDLQRLDGMGCLEFDEERAQQEDALAQQAFEEARRRT
REFEDRDRSHREEMEARRQQDPSPGSNLGGGDDLKLR

The transcription control component of the transcription factor of the present invention may comprise one of the transcription factors shown as SEQ ID No. 7 to 22 or a variant thereof. A variant transcription factor, may have at least 70%, 80%, 90%, 95% or 99% sequence identity to one of the sequences shown as SEQ 7 to 22 as long as it retains the function of the wild-type sequence, i.e. the capacity to up- or down-regulate the transcription of one or more target genes.

First Binding Domain, Second Binding Domain and Agent

The first binding domain, second binding domain and agent of the transcription system of the invention may be any combination of molecules/peptides/domains which enable the selective co-localization and dimerization of the docking component and transcription control component in the absence of the agent.

As such, the first binding domain and second binding domain are capable of specifically binding.

The transcription system of the present invention is not limited by the arrangement of a specific dimerization system. The docking component may comprise either the first binding domain or the second binding domain of a given dimerization system so long as the transcription control component comprises the corresponding, complementary binding domain which enables the docking component and transcription control component to co-localize in the absence of the agent.

The first binding domain and second binding domain may be a peptide domain and a peptide binding domain; or vice versa. The peptide domain and peptide binding domain may be any combination of peptides/domains which are capable of specific binding.

The agent may be a molecule, for example a small molecule, which is capable of specifically binding to the first binding domain or the second binding domain at a higher affinity than the binding between the first binding domain and the second binding domain.

For example, the binding system may be based on a peptide:peptide binding domain system. The first or second binding domain may comprise the peptide binding domain and the other binding domain may comprise a peptide mimic which binds the peptide binding domain with lower affinity than the peptide. The use of peptide as agent disrupts the binding of the peptide mimic to the peptide binding domain through competitive binding. The peptide mimic may have a similar amino acid sequence to the “wild-type” peptide, but with one of more amino acid changes to reduce binding affinity for the peptide binding domain.

For example, the agent may bind the first binding domain or the second binding domain with at least 10, 20, 50, 100, 1000 or 10000-fold greater affinity than the affinity between the first binding domain and the second binding domain.

The agent may be any pharmaceutically acceptable molecule which preferentially binds the first binding domain or the second binding domain with a higher affinity than the affinity between the first binding domain and the second binding domain.

The agent is capable of being delivered to the cytoplasm of a target cell and being available for intracellular binding.

The agent may be capable of crossing the blood-brain barrier.

Small molecule systems for controlling the co-localization of peptides are known in the art, for example the Tet repressor (TetR), TetR interacting protein (TiP), tetracycline system (Klotzsche et al.; J. Biol. Chem. 280, 24591-24599 (2005); Luckner et al.; J. Mol. Biol. 368, 780-790 (2007)).

The Tet Repressor (TetR) System

The Tet operon is a well-known biological operon which has been adapted for use in mammalian cells. The TetR binds tetracycline as a homodimer and undergoes a conformational change which then modulates the DNA binding of the TetR molecules. Klotzsche et al. (as above), described a phage-display derived peptide which activates the TetR. This protein (TetR interacting protein/TiP) has a binding site in TetR which overlaps, but is not identical to, the tetracycline binding site (Luckner et aL; as above). Thus TiP and tetracycline compete for binding of TetR.

In the transcription system of the invention the first binding domain of the docking component may be TetR or TiP, providing that the second binding domain of the transcription control component is the corresponding, complementary binding partner. For example if the first binding domain of the docking component is TetR, the second binding domain of the transcription control component is TiP. If the first binding domain of the docking component is TiP, the second binding domain of the transcription control component is TetR.

For example, the first binding domain or second binding domain may comprise the sequence shown as SEQ ID NO: 23 or SEQ ID NO: 24:

SEQ ID NO: 23-TetR
MSRLDKSKVINSALELLNEVGIEGLTTRKLAQKLGVEQPTLYWHVKNKRA
LLDALAIEMLDRHHTHFCPLEGESWQDFLRNNAKSFRCALLSHRDGAKVH
LGTRPTEKQYETLENQLAFLCQQGFSLENALYALSAVGH
SEQ ID NO: 24-TiP
MWTWNAYAFAAPSGGGS

TetR must homodimerize in order to function. Thus when the first binding domain on the receptor component is TetR, the receptor component may comprise a linker between the transmembrane domain and the first binding domain (TetR). The linker enables TetR to homodimerize with a TetR from a neighbouring receptor component and orient in the correct direction.

The linker may be the sequence shown as SEQ ID NO: 25.

SEQ ID NO: 25-modified CD4 endodomain
ALIVLGGVAGLLLFIGLGIFFCVRCRHRRRQAERMAQIKRVVSEKKTAQA
PHRFQKTCSPI

The linker may alternatively comprise an alternative linker sequence which has similar length and/or domain spacing properties as the sequence shown as SEQ ID NO: 25.

The linker may have at least 80%, 85%, 90%, 95%, 98% or 99% sequence identity to SEQ ID NO: 25 providing it provides the function of enabling TetR to homodimerize with a TetR from a neighbouring receptor component and orient in the correct direction.

One potential disadvantage of the TetR/TiP system is TetR is xenogenic and immunogenic. The TetR sequence may therefore be a variant which is less immunogenic but retains the ability to specifically bind TiP.

Where the first and second binding domains are TetR or TiP or a variant thereof, the agent may be tetracycline, doxycycline, minocycline or an analogue thereof.

An analogue refers to a variant of tetracycline, doxycycline or minocycline which retains the ability to specifically bind to TetR.

Other combinations of binding domains and agents which may be used in the present CAR system are known in the art. For example, the CAR system may use a streptavidin/biotin-based binding system.

Streptavidin-Binding Epitope

The first or second binding domain may comprise one or more streptavidin-binding epitope(s). The other binding domain may comprise a biotin mimic.

Streptavidin is a 52.8 kDa protein from the bacterium Streptomyces avidinii. Streptavidin homo-tetramers have a very high affinity for biotin (vitamin B7 or vitamin H), with a dissociation constant (Kd) ˜10⁻¹⁵ M. The biotin mimic has a lower affinity for streptavidin than wild-type biotin, so that biotin itself can be used as the agent to disrupt or prevent heterodimerisation between the streptavidin domain and the biotin mimic domain. The biotin mimic may bind streptavidin with for example with a Kd of 1 nM to 100 uM.

The ‘biotin mimic’ domain may, for example, comprise a short peptide sequence (for example 6 to 20, 6 to 18, 8 to 18 or 8 to 15 amino acids) which specifically binds to streptavidin.

The biotin mimic may comprise a sequence as shown in Table 1.

TABLE 1
Biotin mimicking peptides.

| name | Sequence | affinity |
|---|---|---|
| Long nanotag | DVEAWLDERVPLVET | 3.6 nM |
| | (SEQ ID NO: 26) | |
| Short nanotag | DVEAWLGAR | 17 nM |
| | (SEQ ID NO: 27) | |
| Streptag | WRHPQFGG | |
| | (SEQ ID NO: 28) | |
| streptagII | WSHPQFEK | 72 uM |
| | (SEQ ID NO: 29) | |
| SBP-tag | MDEKTTGWRGGHVVEGLAG | 2.5 nM |
| | ELEQLRARLEHHPQGQREP | |
| | (SEQ ID NO: 30) | |
| ccstreptag | CHPQGPPC | 230 nM |
| | (SEQ ID NO: 31) | |
| flankedccstreptag | AECHPQGPPCIEGRK | |
| | (SEQ ID NO: 32) | |

The biotin mimic may be selected from the following group: Streptag II, Flankedccstreptag and ccstreptag.

The streptavidin domain may comprise streptavidin having the sequence shown as SEQ ID No. 33 or a fragment or variant thereof which retains the ability to bind biotin. Full length Streptavidin has 159 amino acids. The N and C termini of the 159 residue full-length protein are processed to give a shorter ‘core’ streptavidin, usually composed of residues 13-139; removal of the N and C termini is necessary for the high biotin-binding affinity.

The sequence of “core” streptavidin (residues 13-139) is shown as SEQ ID No. 33.

SEQ ID No. 33
EAGITGTWYNQLGSTFIVTAGADGALTGTYESAVGNAESRYVLTGRYDSA
PATDGSGTALGWTVAWKNNYRNAHSATTWSGQYVGGAEARINTQWLLTSG
TTEANAWKSTLVGHDTFTKVKPSAAS

Streptavidin exists in nature as a homo-tetramer. The secondary structure of a streptavidin monomer is composed of eight antiparallel β-strands, which fold to give an antiparallel beta barrel tertiary structure. A biotin binding-site is located at one end of each β-barrel. Four identical streptavidin monomers (i.e. four identical β-barrels) associate to give streptavidin's tetrameric quaternary structure. The biotin binding-site in each barrel consists of residues from the interior of the barrel, together with a conserved Trp120 from neighbouring subunit. In this way, each subunit contributes to the binding site on the neighbouring subunit, and so the tetramer can also be considered a dimer of functional dimers.

The streptavidin domain of the CAR system of the present invention may consist essentially of a streptavidin monomer, dimer or tetramer.

The sequence of the streptavidin monomer, dimer or tetramer may comprise all or part of the sequence shown as SEQ ID No. 33, or a variant thereof which retains the capacity to bind biotin.

A variant streptavidin sequence may have at least 70, 80, 90, 95 or 99% identity to SEQ ID No. 33 or a functional portion thereof. Variant streptavidin may comprise one or more of the following amino acids, which are involved in biotin binding: residues Asn23, Tyr43, Ser27, Ser45, Asn49, Ser88, Thr90 and Asp128. Variany streptavidin may, for example, comprise all 8 of these residues. Where variant streptavidin is present in the binding domain as a dimer or teramer, it may also comprise Trp120 which is involved in biotin binding by the neighbouring subunit.

Small molecules agents which disrupt protein-protein interactions have long been developed for pharmaceutical purpose (reviewed by Vassilev et al; Small-Molecule Inhibitors of Protein-Protein Interactions ISBN: 978-3-642-17082-9). A transcription system as described may use such a small molecule. The proteins or peptides whose interaction is disrupted (or relevant fragments of these proteins) can be used as the first and/or second binding domains and the small molecule may be used as the agent which switches on or off transcription factor-mediated control of gene expression. Such a system may be varied by altering the small molecule and proteins such the system functions as described but the small molecule is devoid of unwanted pharmacological activity (e.g. in a manner similar to that described by Rivera et al (Nature Med; 1996; 2; 1028-1032)).

A list of proteins/peptides whose interaction is disruptable using an agent such as a small molecule is given in Table 2. These disputable protein-protein interactions (PPI) may be used in the transcription system of the present invention. Further information on these PPIs is available from White et al 2008 (Expert Rev. Mol. Med. 10:e8).

TABLE 2

| Interacting Protein 1 | Interacting Protein 2 | Inhibitor of PPI |
|---|---|---|
| p53 | MDM2 | Nutlin |
| Anti-apoptotic Bcl2 | Apoptotic | GX015 and ABT-737 |
| member | Bcl2 member | |
| Caspase-3, -7 or -9 | X-linked inhibitor of | DIABLO and DIABLO |
| | apoptosis protein (XIAP) | mimetics |
| RAS | RAF | Furano-indene derivative |
| FR2-7 | PD2 domain of DVL | FJ9 |
| T-cell factor (TCF) | Cyclic AMP response | ICG-001 |
| | element binding protein | |
| | (CBP) | |

Second binding domains which competitively bind to the same first binding domain as the agents described above, and thus may be used to co-localise the docking component and the transcription control component of the transcription system in the absence of the agent, may be identified using techniques and methods which are well known in the art. For example such second binding domains may be identified by display of a single domain VHH library.

The first binding domain and/or second binding domain of the transcription system may comprise a variant(s) which is able to specifically bind to the reciprocal binding domain and thus facilitate co-localisation of the docking component and transcription control component.

Variant sequences may have at least 80%, 85%, 90%, 95%, 98% or 99% sequence identity to the wild-type sequence, provided that the sequences provide an effective dimerization system. That is, provided that the sequences facilitate sufficient co-localisation of the docking and trancription components such that they can heterodimerize in the absence of the agent.

The present invention also relates to a method for disrupting the transcription system of the first aspect of the invention, which method comprises the step of administering the agent. As described above, administration of the agent results in a disruption of the co-localization between the docking component and the transcription control component.

The first and second binding domains may control gene expression through the transcription system in a manner which is proportional to the concentration of the agent which is present. Thus, whilst the agent binds the first binding domain or the second binding domain with a higher affinity than binding affinity between the first and second binding domains, co-localization of the docking and transcription control components may not be completely ablated in the presence of low concentrations of the agent. For example, low concentrations of the agent may decrease the total level of gene transcription without completely inhibiting it. The specific concentrations of agent will differ depending on the level of gene transcription required and the specific binding domains and agent.

### Chimeric Antigen Receptor (CAR)

CHIMERIC ANTIGEN RECEPTOR (CAR)

The cell of the present invention may also express a chimeric antigen receptor (CAR).

A classical CAR is a chimeric type I trans-membrane protein which connects an extracellular antigen-recognizing domain (binder) to an intracellular signalling domain (endodomain). The binder is typically a single-chain variable fragment (scFv) derived from a monoclonal antibody (mAb), but it can be based on other formats which comprise an antibody-like antigen binding site. A spacer domain is usually necessary to isolate the binder from the membrane and to allow it a suitable orientation. A common spacer domain used is the Fc of IgG1. More compact spacers can suffice e.g. the stalk from CD8a and even just the IgG1 hinge alone, depending on the antigen. A trans-membrane domain anchors the protein in the cell membrane and connects the spacer to the endodomain.

Early CAR designs had endodomains derived from the intracellular parts of either the γ chain of the FcεR1 or CD3ζ. Consequently, these first generation receptors transmitted immunological signal 1, which was sufficient to trigger T-cell killing of cognate target cells but failed to fully activate the T-cell to proliferate and survive. To overcome this limitation, compound endodomains have been constructed: fusion of the intracellular part of a T-cell co-stimlatory molecule to that of CD3ζ results in second generation receptors which can transmit an activating and co-stimulatory signal simultaneously after antigen recognition. The co-stimulatory domain most commonly used is that of CD28. This supplies the most potent co-stimulatory signal—namely immunological signal 2, which triggers T-cell proliferation. Some receptors have also been described which include TNF receptor family endodomains, such as the closely related OX40 and 41BB which transmit survival signals. Even more potent third generation CARs have now been described which have endodomains capable of transmitting activation, proliferation and survival signals.

CAR-encoding nucleic acids may be transferred to T cells using, for example, retroviral vectors. Lentiviral vectors may be employed. In this way, a large number of cancer-specific T cells can be generated for adoptive cell transfer. When the CAR binds the target-antigen, this results in the transmissin of anctivating signal to the T-cell it is expressed on. Thus the CAR directs the specificity and cytotoxicity of the T cell towards tumour cells expressing the targeted antigen.

CARs typically therefore comprise: (i) an antigen-binding domain; (ii) a spacer; (iii) a transmembrane domain; and (iii) an intracellular domain which comprises or associates with a signalling domain.

Antigen Binding Domain

The antigen binding domain is the portion of the CAR which recognizes antigen. Numerous antigen-binding domains are known in the art, including those based on the antigen binding site of an antibody, antibody mimetics, and T-cell receptors. For example, the antigen-binding domain may comprise: a single-chain variable fragment (scFv) derived from a monoclonal antibody; a natural ligand of the target antigen; a peptide with sufficient affinity for the target; a single domain antibody; an artificial single binder such as a Darpin (designed ankyrin repeat protein); or a single-chain derived from a T-cell receptor.

The antigen binding domain may comprise a domain which is not based on the antigen binding site of an antibody. For example the antigen binding domain may comprise a domain based on a protein/peptide which is a soluble ligand for a tumour cell surface receptor (e.g. a soluble peptide such as a cytokine or a chemokine); or an extracellular domain of a membrane anchored ligand or a receptor for which the binding pair counterpart is expressed on the tumour cell.

The antigen binding domain may be based on a natural ligand of the antigen.

The antigen binding domain may comprise an affinity peptide from a combinatorial library or a de novo designed affinity protein/peptide.

### Spacer Domain

CARs comprise a spacer sequence to connect the antigen-binding domain with the transmembrane domain and spatially separate the antigen-binding domain from the endodomain. A flexible spacer allows the antigen-binding domain to orient in different directions to facilitate binding.

Transmembrane Domain

The transmembrane domain is the sequence of the CAR that spans the membrane.

A transmembrane domain may be any protein structure which is thermodynamically stable in a membrane. This is typically an alpha helix comprising of several hydrophobic residues. The transmembrane domain of any transmembrane protein can be used to supply the transmembrane portion of the invention. The presence and span of a transmembrane domain of a protein can be determined by those skilled in the art using the TMHMM algorithm (http://www.cbs.dtu.dk/services/TMHMM-2.0/). Further, given that the transmembrane domain of a protein is a relatively simple structure, i.e a polypeptide sequence predicted to form a hydrophobic alpha helix of sufficient length to span the membrane, an artificially designed TM domain may also be used (U.S. Pat. No. 7,052,906 B1 describes synthetic transmembrane components).

The transmembrane domain may be derived from CD28, which gives good receptor stability.

Endodomain

The endodomain is the signal-transmission portion of the CAR. It may be part of or associate with the intracellular domain of the CAR. After antigen recognition, receptors cluster, native CD45 and CD148 are excluded from the synapse and a signal is transmitted to the cell. The most commonly used endodomain component is that of CD3-zeta which contains 3 ITAMs. This transmits an activation signal to the T cell after antigen is bound. CD3-zeta may not provide a fully competent activation signal and additional co-stimulatory signaling may be needed. For example, chimeric CD28 and OX40 can be used with CD3-Zeta to transmit a proliferative/survival signal, or all three can be used together.

The endodomain of the CAR or TanCAR of the present invention may comprise the CD28 endodomain and OX40 and CD3-Zeta endodomain.

The endodomain may comprise:

(i) an ITAM-containing endodomain, such as the endodomain from CD3 zeta; and/or

(ii) a co-stimulatory domain, such as the endodomain from CD28; and/or

(iii) a domain which transmits a survival signal, for example a TNF receptor family endodomain such as OX-40 or 4-1 BB.

A number of systems have been described in which the antigen recognition portion is on a separate molecule from the signal transmission portion, such as those described in WO015/150771; WO2016/124930 and WO2016/030691. The CAR expressed by the cell of the present invention may therefore comprise an antigen-binding component comprising an antigen-binding domain and a transmembrane domain; which is capable of interacting with a separate intracellular signalling component comprising a signalling domain. The cell of the invention may comprise a CAR signalling system comprising such an antigen-binding component and intracellular signalling component.

### Signal Peptide

The cell of the present invention may comprise a signal peptide so that when the CAR is expressed inside a cell, the nascent protein is directed to the endoplasmic reticulum and subsequently to the cell surface, where it is expressed.

The signal peptide may be at the amino terminus of the molecule.

The CAR of the invention may have the general formula:

Signal peptide-antigen binding domain-spacer domain-transmembrane domain-intracellular T cell signaling domain (endodomain).

Nucleic Acid Sequence

As used herein, the terms “polynucleotide”, “nucleotide”, and “nucleic acid” are intended to be synonymous with each other.

It will be understood by a skilled person that numerous different polynucleotides and nucleic acid sequences can encode the same polypeptide as a result of the degeneracy of the genetic code. In addition, it is to be understood that skilled persons may, using routine techniques, make nucleotide substitutions that do not affect the polypeptide sequence encoded by the polynucleotides described here to reflect the codon usage of any particular host organism in which the polypeptides are to be expressed.

Nucleic acids according to the invention may comprise DNA or RNA. They may be single-stranded or double-stranded. They may also be polynucleotides which include within them synthetic or modified nucleotides. A number of different types of modification to oligonucleotides are known in the art. These include methylphosphonate and phosphorothioate backbones, addition of acridine or polylysine chains at the 3′ and/or 5′ ends of the molecule. For the purposes of the use as described herein, it is to be understood that the polynucleotides may be modified by any method available in the art. Such modifications may be carried out in order to enhance the in vivo activity or life span of polynucleotides of interest.

The terms “variant”, “homologue” or “derivative” in relation to a nucleotide sequence include any substitution of, variation of, modification of, replacement of, deletion of or addition of one (or more) nucleic acid from or to the sequence.

Nucleic Acid Construct

The present invention provides a nucleic acid construct which comprises a first nucleic acid sequence encoding a docking component and a second nucleic acid sequence encoding a transcription control component as defined above.

The nucleic acid sequences may be in either order in the nucleic acid construct, i.e. first-second; or second-first.

The nucleic acid construct may the following structure:

DC-coexpr-TCC; or

TCC-coexpr-DC

in which:

DC is a nucleic acid sequence encoding the docking component;

coexpr is a nucleic acid sequence enabling co-expression of the docking component and the transcription control component; and

TCC is a nucleic acid sequence encoding the transcription control component.

The nucleic acid construct may also comprise a third nucleic acid sequence encoding a chimeric antigen receptor.

The first, second and third nucleic acids may be in any order in the nucleic acid construct, i.e. 1-2-3, 1-3-2, 2-1-3, 2-3-1, 3-1-2, or 3-2-1.

The nucleic acid construct may, for example, have one of the following structures:

CAR-coexpr1 -DC-coexpr2-TCC;

CAR-coexpr1 -TCC-coexpr2-DC;

DC-coexpr1-TCC-coexpr2-CAR; or

TCC-coexpr1-DC-coexp2-CAR

in which:

CAR is a nucleic acid sequence encoding a chimeric antigen receptor;

DC is a nucleic acid sequence encoding the docking component;

Coexpr1 and coexpr2, which may be the same or different, are nucleic acid sequences enabling co-expression of the docking component, the transcription control component and the chimeric antigen receptor; and

TCC is a nucleic acid sequence encoding the transcription control component.

The nucleic acid construct may also comprise a nucleic acid sequence enabling expression of two or more proteins. For example, it may comprise a sequence encoding a cleavage site between the two nucleic acid sequences. The cleavage site may be self-cleaving, such that when the nascent polypeptide is produced, it is immediately cleaved into the two proteins without the need for any external cleavage activity.

Various self-cleaving sites are known, including the Foot-and-Mouth disease virus (FMDV) 2a self-cleaving peptide, which has the sequence:

SEQ ID NO: 34
RAEGRGSLLTCGDVEENPGP
or
SEQ ID NO: 35
QCTNYALLKLAGDVESNPGP

The co-expressing sequence may alternatively be an internal ribosome entry sequence (IRES) or an internal promoter.

Vector

The present invention also provides a vector, or kit of vectors which comprises one or more nucleic acid sequence(s) or construct(s) according to the present invention. Such a vector may be used to introduce the nucleic acid sequence(s) or construct(s) into a host cell so that it expresses the proteins encoded by the nucleic acid sequence or construct.

The vector may, for example, be a plasmid or a viral vector, such as a retroviral vector or a lentiviral vector, or a transposon based vector or synthetic mRNA.

The vector may be capable of transfecting or transducing a T cell.

Kits

The present invention also provides a kit of nucleic acid sequences which comprises a first nucleic acid sequence encoding a docking component and a second nucleic acid sequence encoding a transcription control component as defined above.

The kit may also comprise a third nucleic acid sequence encoding a chimeric antigen receptor.

The present invention also provides a kit of vectors which comprises a first vector comprising a first nucleic acid sequence encoding a docking component; and a second vector comprising a second nucleic acid sequence encoding a transcription control component as defined above.

The kit may also comprise a third vector comprising a third nucleic acid sequence encoding a chimeric antigen receptor.

The kit of nucleic acid sequences or the kit of vectors may also comprise an agent which causes dissociation of the docking and transcription control components.

Cell

The present invention provides a cell which expresses a transcription system according to the present invention. The cell may also express a chimeric antigen receptor.

The cell may be a cytolytic immune cell.

Cytolytic immune cells can be T cells or T lymphocytes which are a type of lymphocyte that play a central role in cell-mediated immunity. They can be distinguished from other lymphocytes, such as B cells and natural killer cells (NK cells), by the presence of a T-cell receptor (TCR) on the cell surface. There are various types of T cell, as summarised below.

Helper T helper cells (TH cells) assist other white blood cells in immunologic processes, including maturation of B cells into plasma cells and memory B cells, and activation of cytotoxic T cells and macrophages. TH cells express CD4 on their surface. TH cells become activated when they are presented with peptide antigens by MHC class II molecules on the surface of antigen presenting cells (APCs). These cells can differentiate into one of several subtypes, including TH1, TH2, TH3, TH17, Th9, or TFH, which secrete different cytokines to facilitate different types of immune responses.

Cytolytic T cells (TC cells, or CTLs) destroy virally infected cells and tumor cells, and are also implicated in transplant rejection. CTLs express the CD8 at their surface. These cells recognize their targets by binding to antigen associated with MHC class I, which is present on the surface of all nucleated cells. Through IL-10, adenosine and other molecules secreted by regulatory T cells, the CD8+ cells can be inactivated to an anergic state, which prevent autoimmune diseases such as experimental autoimmune encephalomyelitis.

Memory T cells are a subset of antigen-specific T cells that persist long-term after an infection has resolved. They quickly expand to large numbers of effector T cells upon re-exposure to their cognate antigen, thus providing the immune system with “memory” against past infections. Memory T cells comprise three subtypes: central memory T cells (TCM cells) and two types of effector memory T cells (TEM cells and TEMRA cells). Memory cells may be either CD4+ or CD8+. Memory T cells typically express the cell surface protein CD45RO.

Regulatory T cells (Treg cells), formerly known as suppressor T cells, are crucial for the maintenance of immunological tolerance. Their major role is to shut down T cell-mediated immunity toward the end of an immune reaction and to suppress auto-reactive T cells that escaped the process of negative selection in the thymus.

Two major classes of CD4+ Treg cells have been described—naturally occurring Treg cells and adaptive Treg cells.

Naturally occurring Treg cells (also known as CD4+CD25+FoxP3+ Treg cells) arise in the thymus and have been linked to interactions between developing T cells with both myeloid (CD11c+) and plasmacytoid (CD123+) dendritic cells that have been activated with TSLP. Naturally occurring Treg cells can be distinguished from other T cells by the presence of an intracellular molecule called FoxP3. Mutations of the FOXP3 gene can prevent regulatory T cell development, causing the fatal autoimmune disease IPEX.

Adaptive Treg cells (also known as Tr1 cells or Th3 cells) may originate during a normal immune response.

Natural Killer Cells (or NK cells) are a type of cytolytic cell which form part of the innate immune system. NK cells provide rapid responses to innate signals from virally infected cells in an MHC independent manner.

NK cells (belonging to the group of innate lymphoid cells) are defined as large granular lymphocytes (LGL) and constitute the third kind of cells differentiated from the common lymphoid progenitor generating B and T lymphocytes. NK cells are known to differentiate and mature in the bone marrow, lymph node, spleen, tonsils and thymus where they then enter into the circulation.

The cells of the invention may be any of the cell types mentioned above.

Cells of the invention may either be created ex vivo either from a patient's own peripheral blood (1st party), or in the setting of a haematopoietic stem cell transplant from donor peripheral blood (2nd party), or peripheral blood from an unconnected donor (3rd party).

Alternatively, the cells may be derived from ex vivo differentiation of inducible progenitor cells or embryonic progenitor cells to, for example, T cells. Alternatively, an immortalized cell line which retains its lytic function and could act as a therapeutic may be used.

In all these embodiments, cells may generated by introducing DNA or RNA coding for the CAR and transcription factor by one of many means including transduction with a viral vector, transfection with DNA or RNA.

The cell of the invention may be an ex vivo cell from a subject. The cell may be from a peripheral blood mononuclear cell (PBMC) sample. Cells may be activated and/or expanded prior to being transduced with nucleic acid sequence or construct of the invention, for example by treatment with an anti-CD3 monoclonal antibody.

The cell of the invention may be made by:
(i) isolation of a cell-containing sample from a subject or other sources listed above; and
(ii) transduction or transfection of the cells with a nucleic acid sequence or construct according to the invention.

Compositions

The present invention also relates to a pharmaceutical composition containing a plurality of cells of the invention. The pharmaceutical composition may additionally comprise a pharmaceutically acceptable carrier, diluent or excipient. The pharmaceutical composition may optionally comprise one or more further pharmaceutically active polypeptides and/or compounds. Such a formulation may, for example, be in a form suitable for intravenous infusion.

The present invention also provides a composition which comprises a plurality of cells of the invention together with the agent which disrupts binding of the first and second binding domains.

Method of Treatment

The cells of the present invention may be capable of killing target cells, such as cancer cells.

The cells of the present invention may be used for the treatment of an infection, such as a viral infection.

The cells of the invention may also be used for the control of pathogenic immune responses, for example in autoimmune diseases, allergies and graft-vs-host rejection.

The cells of the invention may be used for the treatment of a cancerous disease, such as bladder cancer, breast cancer, colon cancer, endometrial cancer, kidney cancer (renal cell), leukemia, lung cancer, melanoma, non-Hodgkin lymphoma, pancreatic cancer, prostate cancer and thyroid cancer.

The cells of the invention may be used to treat: cancers of the oral cavity and pharynx which includes cancer of the tongue, mouth and pharynx; cancers of the digestive system which includes oesophageal, gastric and colorectal cancers; cancers of the liver and biliary tree which includes hepatocellular carcinomas and cholangiocarcinomas; cancers of the respiratory system which includes bronchogenic cancers and cancers of the larynx; cancers of bone and joints which includes osteosarcoma; cancers of the skin which includes melanoma; breast cancer; cancers of the genital tract which include uterine, ovarian and cervical cancer in women, prostate and testicular cancer in men; cancers of the renal tract which include renal cell carcinoma and transitional cell carcinomas of the utterers or bladder; brain cancers including gliomas, glioblastoma multiforme and medullobastomas; cancers of the endocrine system including thyroid cancer, adrenal carcinoma and cancers associated with multiple endocrine neoplasm syndromes; lymphomas including Hodgkin's lymphoma and non-Hodgkin lymphoma; Multiple Myeloma and plasmacytomas; leukaemias both acute and chronic, myeloid or lymphoid; and cancers of other and unspecified sites including neuroblastoma.

Regulating Gene Transcription

There is also provided a method for regulating the transcription of a gene in a cell of the invention by administering the agent to the cell in vitro.

In the first embodiment of the invention, administration of the agent causes transcription factor mediated gene regulation to be turned on; whereas in the second embodiment of the invention, administration of the agent causes transcription factor mediated gene regulation to be turned off.

The agent may also be used to regulate gene transcription in vivo, by administration of the agent to a subject comprising cells according to the invention. The agent may be administered to the subject before or after or at the same time as administration of cells according to invention to the subject.

The transcription factor turned on or off by the transcription system of the present invention may be involved in controlling T-cell differentiation and/or exhaustion in vivo. In such a case, the agent may be used in vivo or in vitro to preventing or reducing T cell differentiation or exhaustion in a cell of the invention.

The invention will now be further described by way of Examples, which are meant to serve to assist one of ordinary skill in the art in carrying out the invention and are not intended in any way to limit the scope of the invention.

### EXAMPLES

### Example 1

### Development of a Transcriptional Control System

In order to test the transcriptional switching, a model system is constructed, where eGFP expression is modulated. This consists of a split cassette comprising: (a) receiving cassette which consists of a GAL4 responsive promotor, eGFP coding sequence and a polyadenylation sequence; and (b1) a transmitting cassette which consists of DNA coding for membrane-tethered TetR co-expressed via a 2A peptide with a synthetic transcription factor which consists of TIP, GAL4 DNA binding domain fused to VP16 fused to a nuclear localization sequence or an alternative transmitting domain; or (b2) which consists of DNA coding for tetR fused to a nuclear localization signal co-expressed by a 2A peptide to TIP/GAL4/VP16 fusion with a nuclear export signal. T-cells are generated with a receiving cassette and either of the transmitting cassettes stably integrated. eGFP is measured by flow-cytometry without tetracycline and at different time-points after exposure to different concentrations of tetracycline.

### Example 2

### Testing the Transcriptional Control System

In order to test utility of this system with a transcription factor which modulates CAR T-cell differentiation, the following tri-cistronic retroviral vector construct is generated. The BACH2 transcription factor is modified so that TIP is attached to its amino terminus. This is co-expressed with membrane tethered TetR by means of a 2A peptide. This in turn is co-expressed with a CD19 CAR again by means of a 2A peptide. T-cells are modified to express this tri-cistronic cassette by means of retroviral transduction. T-cells are cultured in the presence or absence of tetracycline during production. Their phenotype is studied after transduction using flow-cytometry with antibody panels which test for T-cell differentiation. The function of CAR T-cells generated either in the absence or presence of tetracycline is also tested in vitro in the absence and presence of tetracycline. Finally, CAR T-cells generated in the absence or presence of tetracycline are tested in NSG mice with a B-cell line (Raji and NALM6 engineered to express firefly Luciferase) xenograft. Mice are either given intraperitoneal tetracycline or are given intraperitoneal carrier. Tumour is followed using bioluminscence imaging. After sacrifice, flow-cytometric analysis is performed of spleen and bone-marrow.

### Example 3

### Chimeric Antigen Receptor (CAR and Transcription Factor (TF) Co-Expression

A bicistronic construct was expressed in BW5 T cells as a single transcript which self-cleaves at the 2A site to yield a chimeric antigen receptor (CAR); and a transcription factor (TF). Control constructs were also generated which lack the 2A site and the transcription factor (“CAR-only”) or lack the CAR and 2A site (“TF-only”).

The CAR was an anti-CD19 CAR comprising an endodomain derived from CD3 zeta and from the co-stimulatory receptor 41BB.

Constructs were tested comprising the transcription factors shown in the following table:

| Transcription factor type | Transcription factor |
|---|---|
| Central memory transcription factors | EOMES |
| | FOXO1 |
| | Runx3 and beta catenin |
| Central memory repressors | BACH2 |

### EXAMPLE 2

### Phenotype Assays

The expression of various CARs on the surface of T cells can influence the memory status of those T cells in the absence of the CAR antigen. In addition, binding of the CAR to its cognate antigen activates the T cells and causes further differentiation from a more nave central memory phenotype to a more differentiated effector memory/effector phenotype. Expression of the appropriate transcription factor/repressor is expected to prevent this CAR-mediated differentiation to varying degrees.

T cells expressing the various CAR-TF combinations, together with the relevant CAR-only and TF-only controls were co-cultured with CD19 positive SKOV3 target cells for 24 hours before recovering and culturing the T cells until day 7. The expression of the following memory markers was analysed by flow cytometry at day 0 of the co-culture and day 7, to see whether cells expressing factors that bias them towards central memory are more nave post-transduction and remain more nave upon stimulation with antigen-bearing target cells.

Memory Markers—CCR7, CD45RA, CD62L, CD27

The data for FOXO1 are shown in FIG. 5. For both CD4+ and CD8+ subpopulations, the co-expression of FOXO1 with the CAR (HD37) gave a greater proportion of nave and central memory cells (CM) at both day 0 and day 7. This indicates that FOXO1 biases the cells towards a naive/central memory phenotype both post-transduction and following co-culture with target cells.

FIG. 6 shows CD27 and CD62L expression data 6 days after a 24 hour co-culture with target cells. The transcription factor EOMES caused significant upregulation of CD27 in both the CD4+ and CD8+ T cell subpopulations. FOXO1 caused upregulation of CD27, especially on CD8+ cells. The transcription factor FOXO1 caused significant upregulation of CD62L on both the CD4+ and CD8+ subpopulations. CD62L is a marker of naive/central memory cells and memory phenotyping for FOX01 correlates with the CD62L levels: more naive and memory cells. CD27 is a marker of everything other than fully differentiated effector cells, so it could be that the EOMES-expressing cells are predominantly a less differentiated effector memory sub-type which do not show significant up-regulation of CD62L.

As shown in FIG. 7, the presence of both Runx3 and CBFbeta caused upregulation of CD62L after transduction (day 0) and 6 days after the 24 hour co-culture.

The data for BACH2 and the BACH2 mutant S520A are shown in FIG. 8. Both BACH2 and BACH2 S520A give an increase in the proportion of nave and central memory cells (CM) at both day 0 and day 7.

In separate assays, T cells expressing the various CAR-TF combinations together with the relevant CAR-only were co-cultured with CD19 positive SupT1 target cells. The expression of the following exhaustion markers was analysed by flow cytometry at day 0 of the co-culture and days 2,4, and 7, to see whether cells express “Exhaustion” markers to a lower degree upon stimulation.

Exhaustion Markers—PD1, Tim3, Lag3

The cells were gated on CAR-expression (via RQR8 transduction marker) and various T cell and T-cell subset markers (CD3 and CD8) depending on the sub-population of interest.

All publications mentioned in the above specification are herein incorporated by reference. Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in molecular biology or related fields are intended to be within the scope of the following claims.

1. A transcription system which comprises:
(a) a docking component which comprises a first binding domain; and
(b) a transcription control component which comprises a transcription factor and a second binding domain which binds the first binding domain of the docking component
wherein binding of the first and second binding domains is disrupted by the presence of an agent, such that in the absence of the agent the docking component and the transcription control component heterodimerize.

2. A transcription system according to claim 1, wherein
the docking component also comprises a membrane localisation domain; and
the transcription component also comprises a nuclear localisation signal
such that when the transcription system is expressed in a cell, in the absence of the agent the transcription component is held on the intracellular side of the plasma membrane; whereas in the presence of the agent the transcription component dissociates from the docking component and translocates to the nucleus where the transcription factor binds DNA and regulates the transcription of a gene.

3. A transcription system according to claim 1, wherein
the docking component also comprises a nuclear localisation signal; and
the transcription component also comprises a nuclear export signal
such that when the transcription system is expressed in a cell, in the absence of the agent the transcription component is held in the nucleus where the transcription factor binds DNA and regulates the transcription of a gene; whereas in the presence of the agent the transcription component dissociates from the docking component and translocates to the cytoplasm.

4.-10. (canceled)

11. A transcription system according to claim 1, wherein the transcription factor prevents or reduces T-cell differentiation and/or exhaustion when expressed in a T-cell.

12.-22. (canceled)

23. A nucleic acid construct encoding a transcription system according to claim 1, which comprises
(a) a first nucleic acid sequence encoding a docking component which comprises a first binding domain; and
(b) a second nucleic acid sequence encoding a transcription control component which comprises a transcription factor and a second binding domain which binds the first binding domain of the docking component
wherein binding of the first and second binding domains is disrupted by the presence of an agent, such that in the absence of the agent the docking component and the transcription control component heterodimerize.

24. (canceled)

25. A nucleic acid construct according to claim 23, which comprises a third nucleic acid sequence encoding a chimeric antigen receptor.

26.-27. (canceled)

28. A kit of nucleic acid sequences which comprises
(a) a first nucleic acid sequence encoding a docking component which comprises a first binding domain; and
(b) a second nucleic acid sequence encoding a transcription control component which comprises a transcription factor and a second binding domain which binds the first binding domain of the docking component
wherein binding of the first and second binding domains is disrupted by the presence of an agent, such that in the absence of the agent the docking component and the transcription control component heterodimerize.

29. (canceled)

30. A vector which comprises a nucleic acid construct according to claim 23.

31. A kit of vectors which comprises (a) a first vector which comprises a first nucleic acid sequence encoding a docking component which comprises a first binding domain; and
(b) a second vector which comprises a second nucleic acid sequence encoding a transcription control component which comprises a transcription factor and a second binding domain which binds the first binding domain of the docking component
wherein binding of the first and second binding domains is disrupted by the presence of an agent, such that in the absence of the agent the docking component and the transcription control component heterodimerize.

32. (canceled)

33. A cell which comprises a transcription system according to claim 1.

34. A cell according to claim 33 which expresses a chimeric antigen receptor.

35. A method for making a cell according to claim 33, which comprises the step of introducing: a nucleic acid construct, a kit of nucleic acid sequences, a vector, or a kit of vectors, into a cell.

36. (canceled)

37. A pharmaceutical composition comprising a plurality of cells according to claim 33.

38. A method for treating and/or preventing a disease, which comprises the step of administering a pharmaceutical composition according to claim 37 to a subject.

39.-42. (canceled)

43. A method for regulating the transcription of a gene in a cell according to claim 33, which comprises the step of administering the agent to the cell in vitro.

44. A method for regulating the transcription of a gene in a cell according to claim 33 in vivo in a subject, which comprises the step of administering the agent to the subject.

45. (canceled)

46. A method for preventing or reducing T cell differentiation or exhaustion in a cell comprising a transcription system according to claim 1, which comprises the step of administering the agent to the cell in vitro.

47. A method for preventing or reducing T cell differentiation or exhaustion in a cell comprising a transcription system according to claim 1 in vivo in a subject, which comprises the step of administering the agent to the subject.

48. (canceled)

49. A composition which comprises a plurality of cells according to claim 33 together with the agent which disrupts binding of the first and second binding domains.


SEQUENCE LISTING (40 sequences)

SEQ ID NO: 1 - protein, 7 aa, Artificial Sequence
  nuclear localization signal (NLS) in SV40 Large T-antigen
PKKKRKV

SEQ ID NO: 2 - protein, 16 aa, Artificial Sequence
  NLS of nucleoplasmin
KRPAATKKAGQAKKKK

SEQ ID NO: 3 - protein, 20 aa, Artificial Sequence
  eGFP fused NLS of Nucleoplasmin
AVKRPAATKKAGQAKKKKLD

SEQ ID NO: 4 - protein, 25 aa, Artificial Sequence
  EGL-13 NLS
MSRRRKANPTKLSENAKKLAKEVEN

SEQ ID NO: 5 - protein, 9 aa, Artificial Sequence
  c-Myc NLS
PAAKRVKLD

SEQ ID NO: 6 - protein, 9 aa, Artificial Sequence
  TUS-protein NLS
KLKIKRPVK

SEQ ID NO: 7 - protein, 706 aa, Homo sapiens
MASPADSCIQFTRHASDVLLNLNRLRSRDILTDVVIVVSREQFRAHKTVLMACSGLFYSIFTDQLKCNLSVINLDPEINPEGFCILLDFMYTSRLNLREGNIMAVMATAMYLQMEHVVDTCRKFIKASEAEMVSAIKPPREEFLNSRMLMPQDIMAYRGREVVENNLPLRSAPGCESRAFAPSLYSGLSTPPASYSMYSHLPVSSLLFSDEEFRDVRMPVANPFPKERALPCDSARPVPGEYSRPTLEVSPNVCHSNIYSPKETIPEEARSDMHYSVAEGLKPAAPSARNAPYFPCDKASKEEERPSSEDEIALHFEPPNAPLNRKGLVSPQSPQKSDCQPNSPTESCSSKNACILQASGSPPAKSPTDPKACNWKKYKFIVLNSLNQNAKPEGPEQAELGRLSPRAYTAPPACQPPMEPENLDLQSPTKLSASGEDSTIPQASRLNNIVNRSMTGSPRSSSESHSPLYMHPPKCTSCGSQSPQHAEMCLHTAGPTFPEEMGETQSEYSDSSCENGAFFCNECDCRFSEEASLKRHTLQTHSDKPYKCDRCQASFRYKGNLASHKTVHTGEKPYRCNICGAQFNRPANLKTHTRIHSGEKPYKCETCGARFVQVAHLRAHVLIHTGEKPYPCEICGTRFRHLQTLKSHLRIHTGEKPYHCEKCNLHFRHKSQLRLHLRQKHGAITNTKVQYRVSATDLPPELPKAC

SEQ ID NO: 8 - protein, 841 aa, Homo sapiens
MSVDEKPDSPMYVYESTVHCTNILLGLNDQRKKDILCDVTLIVERKEFRAHRAVLAACSEYFWQALVGQTKNDLVVSLPEEVTARGFGPLLQFAYTAKLLLSRENIREVIRCAEFLRMHNLEDSCFSFLQTQLLNSEDGLFVCRKDAACQRPHEDCENSAGEEEDEEEETMDSETAKMACPRDQMLPEPISFEAAAIPVAEKEEALLPEPDVPTDTKESSEKDALTQYPRYKKYQLACTKNVYNASSHSTSGFASTFREDNSSNSLKPGLARGQIKSEPPSEENEEESITLCLSGDEPDAKDRAGDVEMDRKQPSPAPTPTAPAGAACLERSRSVASPSCLRSLFSITKSVELSGLPSTSQQHFARSPACPFDKGITQGDLKTDYTPFTGNYGQPHVGQKEVSNFTMGSPLRGPGLEALCKQEGELDRRSVIFSSSACDQVSTSVHSYSGVSSLDKDLSEPVPKGLWVGAGQSLPSSQAYSHGGLMADHLPGRMRPNTSCPVPIKVCPRSPPLETRTRTSSSCSSYSYAEDGSGGSPCSLPLCEFSSSPCSQGARFLATEHQEPGLMGDGMYNQVRPQIKCEQSYGTNSSDESGSFSEADSESCPVQDRGQEVKLPFPVDQITDLPRNDFQMMIKMHKLTSEQLEFIHDVRRRSKNRIAAQRCRKRKLDCIQNLECEIRKLVCEKEKLLSERNQLKACMGELLDNFSCLSQEVCRDIQSPEQIQALHRYCPVLRPMDLPTASSINPAPLGAEQNIAASQCAVGENVPCCLEPGAAPPGPPWAPSNTSENCTSGRRLEGTDPGTFSERGPPLEPRSQTVTVDFCQEMTDKCTTDEQPRKDYT

SEQ ID NO: 9 - protein, 841 aa, Artificial Sequence
  mutant Bach2 sequence
MSVDEKPDSPMYVYESTVHCTNILLGLNDQRKKDILCDVTLIVERKEFRAHRAVLAACSEYFWQALVGQTKNDLVVSLPEEVTARGFGPLLQFAYTAKLLLSRENIREVIRCAEFLRMHNLEDSCFSFLQTQLLNSEDGLFVCRKDAACQRPHEDCENSAGEEEDEEEETMDSETAKMACPRDQMLPEPISFEAAAIPVAEKEEALLPEPDVPTDTKESSEKDALTQYPRYKKYQLACTKNVYNASSHSTSGFASTFREDNSSNSLKPGLARGQIKSEPPSEENEEESITLCLSGDEPDAKDRAGDVEMDRKQPSPAPTPTAPAGAACLERSRSVASPSCLRSLFSITKSVELSGLPSTSQQHFARSPACPFDKGITQGDLKTDYTPFTGNYGQPHVGQKEVSNFTMGSPLRGPGLEALCKQEGELDRRSVIFSSSACDQVSTSVHSYSGVSSLDKDLSEPVPKGLWVGAGQSLPSSQAYSHGGLMADHLPGRMRPNTSCPVPIKVCPRSPPLETRTRTSASCSSYSYAEDGSGGSPCSLPLCEFSSSPCSQGARFLATEHQEPGLMGDGMYNQVRPQIKCEQSYGTNSSDESGSFSEADSESCPVQDRGQEVKLPFPVDQITDLPRNDFQMMIKMHKLTSEQLEFIHDVRRRSKNRIAAQRCRKRKLDCIQNLECEIRKLVCEKEKLLSERNQLKACMGELLDNFSCLSQEVCRDIQSPEQIQALHRYCPVLRPMDLPTASSINPAPLGAEQNIAASQCAVGENVPCCLEPGAAPPGPPWAPSNTSENCTSGRRLEGTDPGTFSERGPPLEPRSQTVTVDFCQEMTDKCTTDEQPRKDYT

SEQ ID NO: 10 - protein, 825 aa, Homo sapiens
MLDICLEKRVGTTLAAPKCNSSTVRFQGLAEGTKGTMKMDMEDADMTLWTEAEFEEKCTYIVNDHPWDSGADGGTSVQAEASLPRNLLFKYATNSEEVIGVMSKEYIPKGTRFGPLIGEIYTNDTVPKNANRKYFWRIYSRGELHHFIDGFNEEKSNWMRYVNPAHSPREQNLAACQNGMNIYFYTIKPIPANQELLVWYCRDFAERLHYPYPGELTMMNLTQTQSSLKQPSTEKNELCPKNVPKREYSVKEILKLDSNPSKGKDLYRSNISPLTSEKDLDDFRRRGSPEMPFYPRVVYPIRAPLPEDFLKASLAYGIERPTYITRSPIPSSTTPSPSARSSPDQSLKSSSPHSSPGNTVSPVGPGSQEHRDSYAYLNASYGTEGLGSYPGYAPLPHLPPAFIPSYNAHYPKFLLPPYGMNCNGLSAVSSMNGINNFGLFPRLCPVYSNLLGGGSLPHPMLNPTSLPSSLPSDGARRLLQPEHPREVLVPAPHSAFSFTGAAASMKDKACSPTSGSPTAGTAATAEHVVQPKATSAAMAAPSSDEAMNLIKNKRNMTGYKTLPYPLKKQNGKIKYECNVCAKTFGQLSNLKVHLRVHSGERPFKCQTCNKGFTQLAHLQKHYLVHTGEKPHECQVCHKRFSSTSNLKTHLRLHSGEKPYQCKVCPAKFTQFVHLKLHKRLHTRERPHKCSQCHKNYIHLCSLKVHLKGNCAAAPAPGLPLEDLTRINEEIEKFDISDNADRLEDVEDDISVISVVEKEILAVVRKEKEETGLKVSLQRNMGNGLLSSGCSLYESSDLPLMKLPPSNPLPLVPVKVKQETVEPMDP

SEQ ID NO: 11 - protein, 686 aa, Homo sapiens
MQLGEQLLVSSVNLPGAHFYPLESARGGSGGSAGHLPSAAPSPQKLDLDKASKKFSGSLSCEAVSGEPAAASAGAPAAMLSDTDAGDAFASAAAVAKPGPPDGRKGSPCGEEELPSAAAAAAAAAAAAAATARYSMDSLSSERYYLQSPGPQGSELAAPCSLFPYQAAAGAPHGPVYPAPNGARYPYGSMLPPGGFPAAVCPPGRAQFGPGAGAGSGAGGSSGGGGGPGTYQYSQGAPLYGPYPGAAAAGSCGGLGGLGVPGSGFRAHVYLCNRPLWLKFHRHQTEMIITKQGRRMFPFLSFNINGLNPTAHYNVFVEVVLADPNHWRFQGGKWVTCGKADNNMQGNKMYVHPESPNTGSHWMRQEISFGKLKLTNNKGANNNNTQMIVLQSLHKYQPRLHIVEVTEDGVEDLNEPSKTQTFTFSETQFIAVTAYQNTDITQLKIDHNPFAKGFRDNYDSSHQIVPGGRYGVQSFFPEPFVNTLPQARYYNGERTVPQTNGLLSPQQSEEVANPPQRWLVTPVQQPGTNKLDISSYESEYTSSTLLPYGIKSLPLQTSHALGYYPDPTFPAMAGWGGRGSYQRKMAAGLPWTSRTSPTVFSEDQLSKEKVKEEIGSSWIETPPSIKSLDSNDSGVYTSACKRRRLSPSNSSNENSPSIKCEDINAEEYSKDTSKGMGGYYAFYTTP

SEQ ID NO: 12 - protein, 655 aa, Homo sapiens
MAEAPQVVEIDPDFEPLPRPRSCTWPLPRPEFSQSNSATSSPAPSGSAAANPDAAAGLPSASAAAVSADFMSNLSLLEESEDFPQAPGSVAAAVAAAAAAAATGGLCGDFQGPEAGCLHPAPPQPPPPGPLSQHPPVPPAAAGPLAGQPRKSSSSRRNAWGNLSYADLITKAIESSAEKRLTLSQIYEWMVKSVPYFKDKGDSNSSAGWKNSIRHNLSLHSKFIRVQNEGTGKSSWWMLNPEGGKSGKSPRRRAASMDNNSKFAKSRSRAAKKKASLQSGQEGAGDSPGSQFSKWPASPGSHSNDDFDNWSTFRPRTSSNASTISGRLSPIMTEQDDLGEGDVHSMVYPPSAAKMASTLPSLSEISNPENMENLLDNLNLLSSPTSLTVSTQSSPGTMMQQTPCYSFAPPNTSLNSPSPNYQKYTYGQSSMSPLPQMPIQTLQDNKSSYGGMSQYNCAPGLLKELLTSDSPPHNDIMTPVDPGVAQPNSRVLGQNVMMGPNSVMSTYGSQASHNKMMNPSSHTHPGHAQQTSAVNGRPLPHTVSTMPHTSGMNRLTQVKTPVQVPLPHPMQMSALGGYSSVSSCNGYGRMGLLHQEKLPSDLDGMFIERLDCDMESIIRNDLMDGDTLDFNFDNVLPNQSFPHSVKTTTHSWVSG

SEQ ID NO: 13 - protein, 415 aa, Homo sapiens
MRIPVDPSTSRRFTPPSPAFPCGGGGGKMGENSGALSAQAAVGPGGRARPEVRSMVDVLADHAGELVRTDSPNFLCSVLPSHWRCNKTLPVAFKVVALGDVPDGTVVTVMAGNDENYSAELRNASAVMKNQVARFNDLRFVGRSGRGKSFTLTITVFTNPTQVATYHRAIKVTVDGPREPRRHRQKLEDQTKPFPDRFGDLERLRMRVTPSTPSPRGSLSTTSHFSSQPQTPIQGTSELNPFSDPRQFDRSFPTLPTLTESRFPDPRMHYPGAMSAAFPYSATPSGTSISSLSVAGMPATSRFHHTYLPPPYPGAPQNQSGPFQANPSPYHLYYGTSSGSYQFSMVAGSSSGGDRSPTRMLASCTSSAASVAAGNLMNPSLGGQSDGVEADGSHSNSPTALSTPGRMDEAVWRPY

SEQ ID NO: 14 - protein, 631 aa, Homo sapiens
MVSKLSQLQTELLAALLESGLSKEALIQALGEPGPYLLAGEGPLDKGESCGGGRGELAELPNGLGETRGSEDETDDDGEDFTPPILKELENLSPEEAAHQKAVVETLLQEDPWRVAKMVKSYLQQHNIPQREVVDTTGLNQSHLSQHLNKGTPMKTQKRAALYTWYVRKQREVAQQFTHAGQGGLIEEPTGDELPTKKGRRNRFKWGPASQQILFQAYERQKNPSKEERETLVEECNRAECIQRGVSPSQAQGLGSNLVTEVRVYNWFANRRKEEAFRHKLAMDTYSGPPPGPGPGPALPAHSSPGLPPPALSPSKVHGVRYGQPATSETAEVPSSSGGPLVTVSTPLHQVSPTGLEPSHSLLSTEAKLVSAAGGPLPPVSTLTALHSLEQTSPGLNQQPQNLIMASLPGVMTIGPGEPASLGPTFTNTGASTLVIGLASTQAQSVPVINSMGSSLTTLQPVQFSQPLHPSYQQPLMPPVQSHVTQSPFMATMAQLQSPHALYSHKPEVAQYTHTGLLPQTMLITDTTNLSALASLTPTKQVFTSDTEASSESGLHTPASQATTLHVPSQDPAGIQHLQPAHRLSASPTVSSSSLVLYQSSDSSNGQSHLLPSNHSVIETFISTQMASSSQ

SEQ ID NO: 15 - protein, 399 aa, Homo sapiens
MPQLSGGGGGGGGDPELCATDEMIPFKDEGDPQKEKIFAEISHPEEEGDLADIKSSLVNESEIIPASNGHEVARQAQTSQEPYHDKAREHPDDGKHPDGGLYNKGPSYSSYSGYIMMPNMNNDPYMSNGSLSPPIPRTSNKVPVVQPSHAVHPLTPLITYSDEHFSPGSHPSHIPSDVNSKQGMSRHPPAPDIPTFYPLSPGGVGQITPPLGWQGQPVYPITGGFRQPYPSSLSVDTSMSRFSHHMIPGPPGPHTTGIPHPAIVTPQVKQEHPHTDSDLMHVKPQHEQRKEQEPKRPHIKKPLNAFMLYMKEMRANVVAECTLKESAAINQILGRRWHALSREEQAKYYELARKERQLHMQLYPGWSARDNYGKKKKRKREKLQESASGTGPRMTAAYI

SEQ ID NO: 16 - protein, 119 aa, Homo sapiens
MKALSPVRGCYEAVCCLSERSLAIARGRGKGPAAEEPLSLLDDMNHCYSRLRELVPGVPRGTQLSQVEILQRVIDYILDLQVVLAEPAPGPPDGPHLPIQTAELTPELVISNDKRSFCH

SEQ ID NO: 17 - protein, 535 aa, Homo sapiens
MGIVEPGCGDMLTGTEPMPGSDEGRAPGADPQHRYFYPEPGAQDADERRGGGSLGSPYPGGALVPAPPSRFLGAYAYPPRPQAAGFPGAGESFPPPADAEGYQPGEGYAAPDPRAGLYPGPREDYALPAGLEVSGKLRVALNNHLLWSKFNQHQTEMIITKQGRRMFPFLSFTVAGLEPTSHYRMFVDVVLVDQHHWRYQSGKWVQCGKAEGSMPGNRLYVHPDSPNTGAHWMRQEVSFGKLKLTNNKGASNNVTQMIVLQSLHKYQPRLHIVEVNDGEPEAACNASNTHIFTFQETQFIAVTAYQNAEITQLKIDNNPFAKGFRENFESMYTSVDTSIPSPPGPNCQFLGGDHYSPLLPNQYPVPSRFYPDLPGQAKDVVPQAYWLGAPRDHSYEAEFRAVSMKPAFLPSAPGPTMSYYRGQEVLAPGAGWPVAPQYPPKMGPASWFRPMRTLPMEPGPGGSEGRGPEDQGPPLVWTEIAPIRPESSDSGLGEGDSKRRRVSPYPSSGDSSSPAGAPSPFDKEAEGQFYNYFPN

SEQ ID NO: 18 - protein, 331 aa, Homo sapiens
MTAKMETTFYDDALNASFLPSESGPYGYSNPKILKQSMTLNLADPVGSLKPHLRAKNSDLLTSPDVGLLKLASPELERLIIQSSNGHITTTPTPTQFLCPKNVTDEQEGFAEGFVRALAELHSQNTLPSVTSAAQPVNGAGMVAPAVASVAGGSGSGGFSASLHSEPPVYANLSNFNPGALSSGGGAPSYGAAGLAFPAQPQQQQQPPHHLPQQMPVQHPRLQALKEEPQTVPEMPGETPPLSPIDMESQERIKAERKRMRNRIAASKCRKRKLERIARLEEKVKTLKAQNSELASTANMLREQVAQLKQKVMNHVNSGCQLMLTQQLQTF

SEQ ID NO: 19 - protein, 134 aa, Homo sapiens
MKAFSPVRSVRKNSLSDHSLGISRSKTPVDDPMSLLYNMNDCYSKLKELVPSIPQNKKVSKMEILQHVIDYILDLQIALDSHPTIVSLHHQRPGQNQASRTPLTTLNTDISILSLQASEFPSELMSNDSKALCG

SEQ ID NO: 20 - protein, 443 aa, Homo sapiens
MEVTADQPRWVSHHHPAVLNGQHPDTHHPGLSHSYMDAAQYPLPEEVDVLFNIDGQGNHVPPYYGNSVRATVQRYPPTHHGSQVCRPPLLHGSLPWLDGGKALGSHHTASPWNLSPFSKTSIHHGSPGPLSVYPPASSSSLSGGHASPHLFTFPPTPPKDVSPDPSLSTPGSAGSARQDEKECLKYQVPLPDSMKLESSHSRGSMTALGGASSSTHHPITTYPPYVPEYSSGLFPPSSLLGGSPTGFGCKSRPKARSSTGRECVNCGATSTPLWRRDGTGHYLCNACGLYHKMNGQNRPLIKPKRRLSAARRAGTSCANCQTTTTTLWRRNANGDPVCNACGLYYKLHNINRPLTMKKEGIQTRNRKMSSKSKKCKKVHDSLEDFPKNSSFNPAALSRHMSSLSHISPFSHSSHMLTTPTPMHPPSSLSFGPHHPSSMVTAMG

SEQ ID NO: 21 - protein, 518 aa, Homo sapiens
MDRAPQRQHRASRELLAAKKTHTSQIEVIPCKICGDKSSGIHYGVITCEGCKGFFRRSQRCNAAYSCTRQQNCPIDRTSRNRCQHCRLQKCLALGMSRDAVKFGRMSKKQRDSLHAEVQKQLQQRQQQQQEPVVKTPPAGAQGADTLTYTLGLPDGQLPLGSSPDLPEASACPPGLLKASGSGPSYSNNLAKAGLNGASCHLEYSPERGKAEGRESFYSTGSQLTPDRCGLRFEEHRHPGLGELGQGPDSYGSPSFRSTPEAPYASLTEIEHLVQSVCKSYRETCQLRLEDLLRQRSNIFSREEVTGYQRKSMWEMWERCAHHLTEAIQYVVEFAKRLSGFMELCQNDQIVLLKAGAMEVVLVRMCRAYNADNRTVFFEGKYGGMELFRALGCSELISSIFDFSHSLSALHFSEDEIALYTALVLINAHRPGLQEKRKVEQLQYNLELAFHHHLCKTHRQSILAKLPPKGKLRSLCSQHVERLQIFQHLHPIVVQAAFPPLYKELFSTETESPVGLSK

SEQ ID NO: 22 - protein, 187 aa, Homo sapiens
MPRVVPDQRSKFENEEFFRKLSRECEIKYTGFRDRPHEERQARFQNACRDGRSEIAFVATGTNLSLQFFPASWQGEQRQTPSREYVDLEREAGKVYLKAPMILNGVCVIWKGWIDLQRLDGMGCLEFDEERAQQEDALAQQAFEEARRRTREFEDRDRSHREEMEARRQQDPSPGSNLGGGDDLKLR

SEQ ID NO: 23 - protein, 139 aa, Artificial Sequence
  TetR binding domain
MSRLDKSKVINSALELLNEVGIEGLTTRKLAQKLGVEQPTLYWHVKNKRALLDALAIEMLDRHHTHFCPLEGESWQDFLRNNAKSFRCALLSHRDGAKVHLGTRPTEKQYETLENQLAFLCQQGFSLENALYALSAVGH

SEQ ID NO: 24 - protein, 17 aa, Artificial Sequence
  TiP binding domain
MWTWNAYAFAAPSGGGS

SEQ ID NO: 25 - protein, 61 aa, Artificial Sequence
  modified CD4 endodomain linker
ALIVLGGVAGLLLFIGLGIFFCVRCRHRRRQAERMAQIKRVVSEKKTAQAPHRFQKTCSPI

SEQ ID NO: 26 - protein, 15 aa, Artificial Sequence
  Biotin mimicking peptide, long nanotag
DVEAWLDERVPLVET

SEQ ID NO: 27 - protein, 9 aa, Artificial Sequence
  Biotin mimicking peptide, short nanotag
DVEAWLGAR

SEQ ID NO: 28 - protein, 8 aa, Artificial Sequence
  Biotin mimicking peptide, streptag
WRHPQFGG

SEQ ID NO: 29 - protein, 8 aa, Artificial Sequence
  Biotin mimicking peptide, streptagII
WSHPQFEK

SEQ ID NO: 30 - protein, 38 aa, Artificial Sequence
  Biotin mimicking peptide, SBP-tag
MDEKTTGWRGGHVVEGLAGELEQLRARLEHHPQGQREP

SEQ ID NO: 31 - protein, 8 aa, Artificial Sequence
  Biotin mimicking peptide, ccstreptag
CHPQGPPC

SEQ ID NO: 32 - protein, 15 aa, Artificial Sequence
  Biotin mimicking peptide, flankedccstreptag
AECHPQGPPCIEGRK

SEQ ID NO: 33 - protein, 126 aa, Artificial Sequence
  core streptavidin sequence
EAGITGTWYNQLGSTFIVTAGADGALTGTYESAVGNAESRYVLTGRYDSAPATDGSGTALGWTVAWKNNYRNAHSATTWSGQYVGGAEARINTQWLLTSGTTEANAWKSTLVGHDTFTKVKPSAAS

SEQ ID NO: 34 - protein, 20 aa, Foot-and-mouth disease virus
RAEGRGSLLTCGDVEENPGP

SEQ ID NO: 35 - protein, 20 aa, Foot-and-mouth disease virus
QCTNYALLKLAGDVESNPGP

SEQ ID NO: 36 - protein, 10 aa, Artificial Sequence
  nuclear export signal (NES), common hydrophobic residues spacing
  MISC_FEATURE (1)..(1): Xaa is a hydrophobic residue (often leucine)
  misc_feature (2)..(4): Xaa can be any naturally occurring amino acid
  MISC_FEATURE (5)..(5): Xaa is a hydrophobic residue (often leucine)
  misc_feature (6)..(7): Xaa can be any naturally occurring amino acid
  MISC_FEATURE (8)..(8): Xaa is a hydrophobic residue (often leucine)
  misc_feature (9)..(9): Xaa can be any naturally occurring amino acid
  MISC_FEATURE (10)..(10): Xaa is a hydrophobic residue (often leucine)
XXXXXXXXXX

SEQ ID NO: 37 - protein, 4 aa, Artificial Sequence
  monopartite NLS consensus sequence
  MISC_FEATURE (2)..(2): Xaa may be Lys or Arg
  misc_feature (3)..(3): Xaa can be any naturally occurring amino acid
  MISC_FEATURE (4)..(4): Xaa may be Lys or Arg
KXXX

SEQ ID NO: 38 - DNA, 10 bp, Artificial Sequence
  C/EBP transcription factor recognition sequence
attgcgcaat

SEQ ID NO: 39 - DNA, 15 bp, Artificial Sequence
  NF-1 transcription factor recognition sequence
  misc_feature (6)..(10): n is a, c, g, or t
ttggcnnnnngccaa

SEQ ID NO: 40 - protein, 7 aa, Artificial Sequence
  consensus sequence of Akt targets
  misc_feature (2)..(2): Xaa can be any naturally occurring amino acid
  misc_feature (4)..(5): Xaa can be any naturally occurring amino acid
  MISC_FEATURE (6)..(6): Xaa may be Ser or Thr
  misc_feature (7)..(7): Xaa can be any naturally occurring amino acid
RXRXXXX